SEQUENCE LISTING (7 sequences)

SEQ ID NO: 1 - protein, 23 aa, Artificial Sequence
  Artificial peptide sequence
RPKHPIKHQGLPQEVLNENLLRF

SEQ ID NO: 2 - protein, 23 aa, Artificial Sequence
  Artificial peptide sequence
INKKIEKFQSEEQQQTEDELQDK

SEQ ID NO: 3 - protein, 23 aa, Artificial Sequence
  Artificial peptide sequence
EKFQSEEQQQTEDELQDKIHPFA

SEQ ID NO: 4 - protein, 23 aa, Artificial Sequence
  Artificial peptide sequence
VMFPPQSVLSLSQSKVLPVPEKA

SEQ ID NO: 5 - protein, 23 aa, Artificial Sequence
  Artificial peptide sequence
PIQAFLLYQEPVLGPVRGPFPII

SEQ ID NO: 6 - protein, 35 aa, Artificial Sequence
  Artificial peptide sequence
RPKHPIKHQGLPQEVLNENLLRFFVAPGPEVFGKE

SEQ ID NO: 7 - protein, 35 aa, Artificial Sequence
  Artificial peptide sequence
INKKIEKFQSEEQQQTEDELQDKIHPFAQTQSLVY

(19) United States
(12) Patent Application Publication
(10) Pub. No.: US 2019/0241936 A1
(43) Pub. Date: Aug. 8, 2019

(54) TEST STRIP FOR MICROORGANISM DETECTION AND DETECTION METHOD USING THE SAME

(71) Applicant: Industrial Technology Research Institute, Hsinchu (TW)

(72) Inventors: Tseng-Huang Liu, Kaohsiung City (TW); Chia-Ying Tang, Hsinchu City (TW); Pei-Shin Jiang, Hsinchu City (TW)

(73) Assignee: Industrial Technology Research Institute, Hsinchu (TW)

(21) Appl. No.: 16/227,735

(22) Filed: Dec. 20, 2018

Related U.S. Application Data
(60) Provisional application No. 62/608,177, filed on Dec. 20, 2017.

(51) Int. Cl. C12Q 1/689; C07K 17/02; C12Q 1/6895; G01N 33/543

(52) U.S. Cl. CPC C12Q 1/689; G01N 33/54306; C12Q 1/6895; C07K 17/02

(57) ABSTRACT

A test strip for microorganism detection includes: a porous substrate, a surface of which a surface has a first color; and a microorganism detection composition, which is at least adhered to a part of a surface of the porous substrate or at least adhered to a part of a surface of the porous substrate and a part of an interior of the porous substrate. The microorganism detection composition includes: a plurality of colored particles having a second color different than the first color; and a plurality of specific peptides immobilized on the surfaces of the plurality of colored particles, in which the plurality of specific peptides connect the plurality of colored particles to each other to make the plurality of colored particles aggregate to cover the part of the surface of the porous substrate and present the second color on the part of the surface of the porous substrate.

### CROSS REFERENCE TO RELATED APPLICATION

This application claims the benefit of U.S. Provisional Application No. 62/608,177, filed on Dec. 20, 2017, the entirety of which is incorporated by reference herein.

### INCORPORATION BY REFERENCE OF SEQUENCE LISTING

A sequence listing submitted as a text file via EFS-Web is incorporated herein by reference. The text file containing the sequence listing is named “0965-A25575-US_Seq_Listing.txt”; its date of creation was Dec. 20, 2018; and its size is 2,775 bytes.

### TECHNICAL FIELD

The present disclosure is related to detection of microorganisms, and it is particularly related to a test strip for microorganism detection and a microorganism detection method.

### BACKGROUND

Traditional microbial detection methods need to be carried out through culturing, biochemical detection, microscopic examination, etc., which are time-consuming and laborious. Moreover, the test samples require pre-treatment steps, but the steps are cumbersome and it is not easy to directly and immediately monitor the state of microorganisms in the environment.

Instruments such as biosensors have been developed to perform real-time monitoring and detection of food microorganisms, and they are used widely, especially to maintain quality control of food. However, the use of such instruments and equipment is restricted to the inspection of random samples, as it is impossible to fully inspect the quality of all products. In addition, instruments and equipment cannot be popularized in practical application due to its high cost.

Therefore, it is extremely necessary to develop a simple, effective and low-cost microbial detection method.

### SUMMARY

The present disclosure provides a test strip for microorganism detection, comprising: a porous substrate, a surface of which has a first color; and a microorganism detection composition which is at least adhered to a part of a surface of the porous substrate or at least adhered to a part of a surface of the porous substrate and a part of an interior of the porous substrate. The microorganism detection composition comprises: a plurality of colored particles having a second color that is different than the first color; and a plurality of specific peptides immobilized on the surfaces of the plurality of colored particles, in which the plurality of specific peptides connect the plurality of colored particles to each other to make the plurality of colored particles aggregate to cover the part of the surface of the porous substrate and present the second color on the part of the surface of the porous substrate. Moreover, the specific peptide is designed for a specific microorganism, and the specific microorganism has the ability to cleave the specific peptide.

The present disclosure also provides a microorganism detection method, comprising bringing a test sample into contact with the test strip for microorganism detection mentioned above; and determining the color change of the test strip for detection after a test sample comes into contact with the test strip for microorganism detection is completed. When the test sample contains the specific microorganism, the specific microorganism cleaves the specific peptide, and that leads to the plurality of colored particles no longer aggregating to lighten the second color, which is present due to the aggregation of the plurality of colored particles, or to expose part of the surface of the porous substrate, thereby making the first color visible.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1A shows a top view of the test strip for microorganism detection 100 of the present disclosure;

FIG. 1B show a sectional view of the structure of the test strip for microorganism detection 100 in one embodiment of the present disclosure;

FIG. 1C show a sectional view of the structure of the test strip for microorganism detection 100 in another embodiment of the present disclosure;

FIG. 2 shows a schematic diagram of the plurality of colored particles being connected to each other by the plurality of specific peptides in the microorganism detection composition;

FIG. 3 is a schematic diagram showing one embodiment of a mode for forming the test strip for microorganism detection 100 of the present disclosure shown in FIG. 1B;

FIGS. 4A-4C are schematic diagrams showing different embodiments of a mode for forming the test strip for microorganism detection 100 of the present disclosure shown in FIG. 1C; microorganism in a test sample by the microorganism detection method of the present disclosure;

FIG. 6 show the results of dropping the magnetic bead suspension on the photocopy paper, the filter paper, the nitrocellulose film and the PVDF film;

FIG. 7 show the results of analyzing the test strips respectively obtained by the two methods recited in Example 3 by a scanning electron microscope;

FIG. 8A shows photographs of the test strip treated with proteinase K with different dilution ratios in an enzymatic degradation test, in which the test strip contains magnetic beads on which α1-23-mer peptide is immobilized;

FIG. 8B shows the digestion levels (DL) of peptide of the test strip treated with proteinase K with different dilution ratios in an enzymatic degradation test, in which the test strip contains magnetic beads on which α1-23-mer peptide is immobilized;

FIG. 9A shows photographs of the test strip treated with trypsin with different dilution ratios in an enzymatic degradation test, in which the test strip contains magnetic beads on which β2-23-mer peptide is immobilized;

FIG. 9B shows the digestion levels of peptide of the test strip treated with trypsin with different dilution ratios in an enzymatic degradation test, in which the test strip contains magnetic beads on which β2-23-mer peptide is immobilized;

FIG. 10A shows photographs of the test strip treated with different lactic acid bacteria in a lactic acid bacteria test, in which the test strip contains magnetic beads on which α1-23-mer peptide is immobilized;

FIG. 10B shows the detachment levels of the test strip treated with different lactic acid bacteria in a lactic acid bacteria test, in which the test strip contains magnetic beads on which α1-23-mer peptide without modification is immobilized;

FIG. 11A shows photographs of the test strip treated with different lactic acid bacteria in a lactic acid bacteria test, in which the test strip contains magnetic beads on which β2-23-mer peptide is immobilized;

FIG. 11B shows the detachment levels of the test strip treated with different lactic acid bacteria in a lactic acid bacteria test, in which the test strip contains magnetic beads on which β2-23-mer peptide without modification is immobilized;

FIG. 12A shows photographs of the test strip treated with proteinase K with different dilution ratios in an enzymatic degradation test, in which the test strip is prepared by item 3.1.2 in Method A of Example 3 and contains magnetic beads on which α1-23-mer-NH₂ peptide is immobilized;

FIG. 12B shows the digestion levels of peptide of the test strip treated with proteinase K with different dilution ratios in an enzymatic degradation test, in which the test strip is prepared by item 3.1.2 in Method A of Example 3 and contains magnetic beads on which α1-23-mer-NH₂ peptide is immobilized;

FIG. 12C shows the area changes of the region covered by the magnetic beads of the test strip treated with proteinase K with different dilution ratios in an enzymatic degradation test, in which the test strip is prepared by item 3.1.2 in Method A of Example 3 and contains magnetic beads on which α1-23-mer-NH₂ peptide is immobilized;

FIG. 12D shows the detachment levels of the test strip treated with proteinase K with different dilution ratios in an enzymatic degradation test, in which the test strip is prepared by item 3.1.2 in Method A of Example 3 and contains magnetic beads on which α1-23-mer-NH₂ peptide is immobilized;

FIG. 13A shows photographs of the test strip treated with trypsin with different dilution ratios in an enzymatic degradation test, in which the test strip is prepared by item 3.1.2 in Method A of Example 3 and contains magnetic beads on which β2-23-mer-NH₂ is immobilized;

FIG. 13B shows the digestion levels of peptide of the test strip treated with trypsin with different dilution ratios in an enzymatic degradation test, in which the test strip is prepared by item 3.1.2 in Method A of Example 3 and contains magnetic beads on which β2-23-mer-NH₂ peptide is immobilized;

FIG. 13C shows the area changes of the region covered by the magnetic beads of the test strip treated with trypsin with different dilution ratios in an enzymatic degradation test, in which the test strip is prepared by item 3.1.2 in Method A of Example 3 and contains magnetic beads on which β2-23-mer-NH₂ peptide is immobilized;

FIG. 13D shows the detachment levels of the test strip treated with trypsin with different dilution ratios in an enzymatic degradation test, in which the test strip is prepared by item 3.1.2 in Method A of Example 3 and contains magnetic beads on which β2-23-mer-NH₂ peptide is immobilized;

FIG. 14 shows photographs of the test strip treated with proteinase K with different dilution ratios in an enzymatic degradation test, in which the test strip is prepared by Method B of Example 3 and contains magnetic beads on which α1-23-mer-NH₂ peptide is immobilized;

FIG. 15 shows photographs of the test strip treated with trypsin with different dilution ratios in an enzymatic degradation test, in which the test strip is prepared by Method B of Example 3 and contains magnetic beads on which β2-23-mer-NH₂ peptide is immobilized;

FIG. 16A shows photographs of the test strip treated with proteinase K with different dilution ratios in an enzymatic degradation test, in which the test strip is prepared by item 3.1.2 in Method A of Example 3 and contains magnetic beads on which α1-35-mer-NH₂ peptide is immobilized;

FIG. 16B shows the detachment levels of the test strip treated with proteinase K with different dilution ratios in an enzymatic degradation test, in which the test strip is prepared by item 3.1.2 in Method A of Example 3 and contains magnetic beads on which α1-35-mer-NH₂ peptide is immobilized;

FIG. 17A shows photographs of the test strip treated with trypsin with different dilution ratios in an enzymatic degradation test, in which the test strip is prepared by item 3.1.2 in Method A of Example 3 and contains magnetic beads on which β2-35-mer-NH₂ peptide is immobilized;

FIG. 17B shows the detachment levels of the test strip treated with trypsin with different dilution ratios in an enzymatic degradation test, in which the test strip is prepared by item 3.1.2 in Method A of Example 3 and contains magnetic beads on which β2-35-mer-NH₂ peptide is immobilized;

FIG. 18A shows photographs of the test strip containing magnetic beads on which α1-23-mer-NH₂ peptide or β2-23-mer-NH₂ peptide is immobilized in a lactic acid bacteria test at a low temperature;

FIG. 18B shows the detachment levels of the test strip containing magnetic beads on which α1-23-mer-NH₂ peptide is immobilized in a lactic acid bacteria test at a low temperature;

FIG. 18C shows the detachment levels of the test strip containing magnetic beads on which β2-23-mer-NH₂ peptide is immobilized in a lactic acid bacteria test at a low temperature;

FIG. 19A shows photographs of the test strip containing magnetic beads on which α1-23-mer-NH₂ peptide is immobilized in a confirmation test for the minimum detectable amount of Lactobacillus plantarum for the test strip; and

FIG. 19B shows the detachment levels of the test strip containing magnetic on which α1-23-mer-NH₂ peptide is immobilized in a confirmation test for the minimum detectable amount of Lactobacillus plantarum for the test strip.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The present disclosure provides a test strip for microorganism detection which is light and thin, and is easy to carry and use. The test strip for microorganism detection of the present disclosure is used for detecting the presence of a specific microorganism. By using the test strip for microorganism detection of the present disclosure, the presence or absence of a specific microorganism can be quickly determined only through visual inspection.

Examples of the specific microorganism mentioned above may comprise a bacterium, a fungi, etc., but they are not limited thereto. The bacterium may comprise, but is not limited to a lactic acid bacterium, Escherichia coli, a bacterium belonging to the genus Salmonella, Staphylococcus aureus, a bacterium belonging to the genus Listeria or a bacterium belonging to the genus Shigella, etc. Moreover, examples of the lactic acid bacterium mentioned above may comprise Lactobacillus reuteri, Lactobacillus pentosus, Lactobacillus gasseri, Lactobacillus salivarius, Lactobacillus paracasei, Lactobacillus plantarum, any combination thereof, but they are not limited thereto.

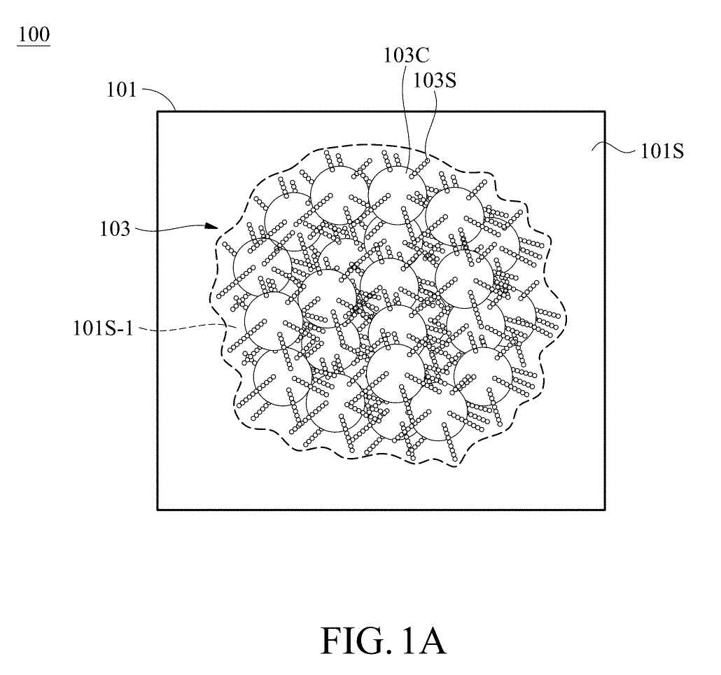

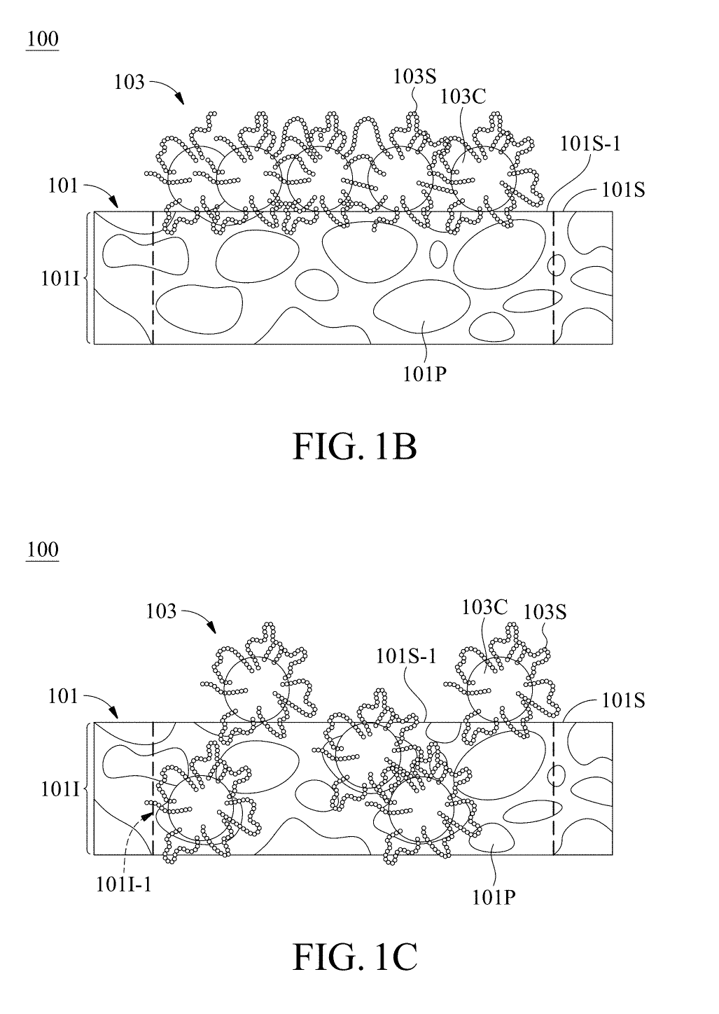

In order make the description more clear, in the following, the composition and structure of the test strip for microorganism detection of the present disclosure is illustrated by FIG. 1 to FIG. 5. However, it should be noted that the test strip for microorganism of present disclosure is not limited to the figures mentioned above.

FIGS. 1A-1C are schematic diagrams of the test strip for microorganism detection 100 of the present disclosure. FIG. 1A shows a top view of the test strip for microorganism detection 100 of the present disclosure. FIG. 1B shows a sectional view of the structure of the test strip for microorganism detection 100 in one embodiment of the present disclosure while FIG. 1C shows a sectional view of the structure of the test strip for microorganism detection 100 in another embodiment of the present disclosure.

Refer to FIGS. 1A-1C. The test strip for microorganism detection 100 of the present disclosure may comprise, but is not limited to a porous substrate 101 and a microorganism detection composition 103. A surface 101S of the foregoing porous substrate 101 has a first color. The microorganism detection composition 103 is at least adhered to a part 101S-1 of a surface 101S of the foregoing porous substrate 101 (referring to FIG. 1B) or at least adhered to a part 101S-1 of a surface 101S of the foregoing porous substrate 101 and a part 101I-1 of an interior 101I of the foregoing porous substrate 101 (referring to FIG. 1C).

Material suited for being used as the material for the porous substrate 101 of the present disclosure may be any porous and has no particular limitation. In one embodiment, the material for the porous substrate 101 may comprise a natural fiber material, an artificial fiber material, etc., but it is not limited thereto. Examples of the natural fiber material mentioned above may comprise, but are not limited to, paper, cotton, hemp, silk, a combination thereof and the like. Examples of the artificial fiber material mentioned above may comprise nitrocellulose, cellulose acetate, polyester, a combination thereof and the like, but they are not limited thereto. In one specific embodiment, the material for the porous substrate 101 may be nitrocellulose.

Furthermore, a pore size of the material for the porous substrate 101 may be 0.01 μm-5.00 μm, such as about 0.01 μm-0.05 μm, about 0.05 μm-1.00 μm, about 1.00 μm-5.00 μm, etc., but it is not limited thereto.

In the test strip for microorganism detection 100 of the present disclosure, microorganism detection composition 103 mentioned above may comprise a plurality of colored particles 103C and a plurality of specific peptides 103S, but it is not limited thereto. The colored particle 103C mentioned above has a second color that is different than the first color. The foregoing plurality of specific peptides 1035 are immobilized on the surfaces of the plurality of colored particles 103C. The foregoing plurality of specific peptides 103S may connect the plurality of colored particles 103C to each other to make the plurality of colored particles 103C aggregate to enable the microorganism detection composition 103 mentioned above cover the part 101S-1 of the surface 101S of the porous substrate 101 to which the microorganism detection composition 103 is adhered to and present the second color on the foregoing part 101S-1 of the surface 101S of the porous substrate 101.

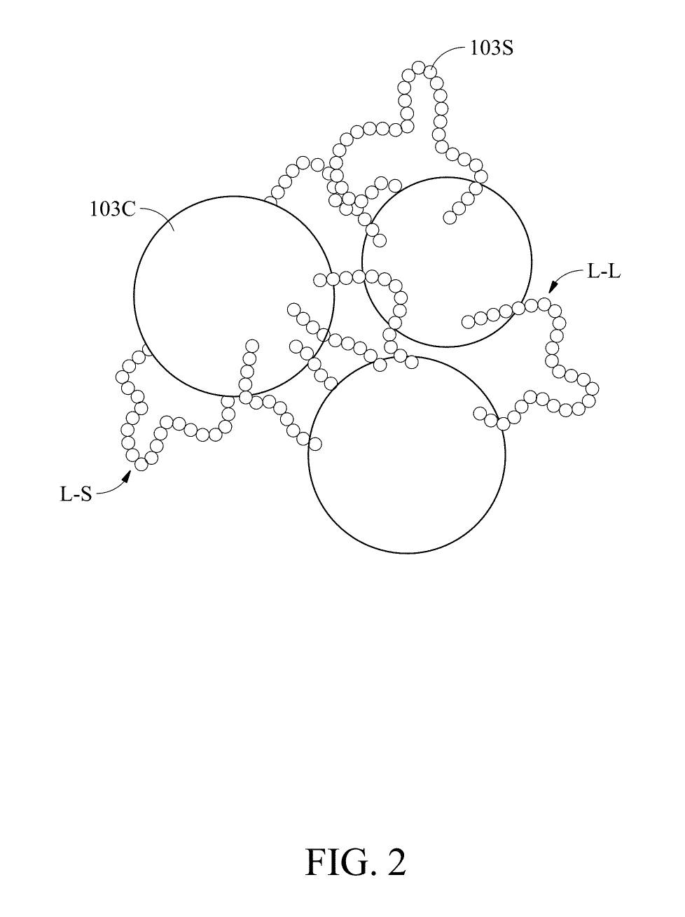

FIG. 2 shows a schematic diagram of the plurality of colored particles 103C being connected to each other by the plurality of specific peptides 103S in the microorganism detection composition 103. According to FIG. 2, it can be known that when the two ends of the specific peptide 103S are respectively immobilized on the surfaces of two different particles of the plurality of colored particles 103C, an inter-link L-L is formed between the two different particles, and when the two ends of the specific peptide 103S are respectively immobilized on the surface of the same particle of the plurality of colored particles 103C, the same particle forms a self-link L-S.

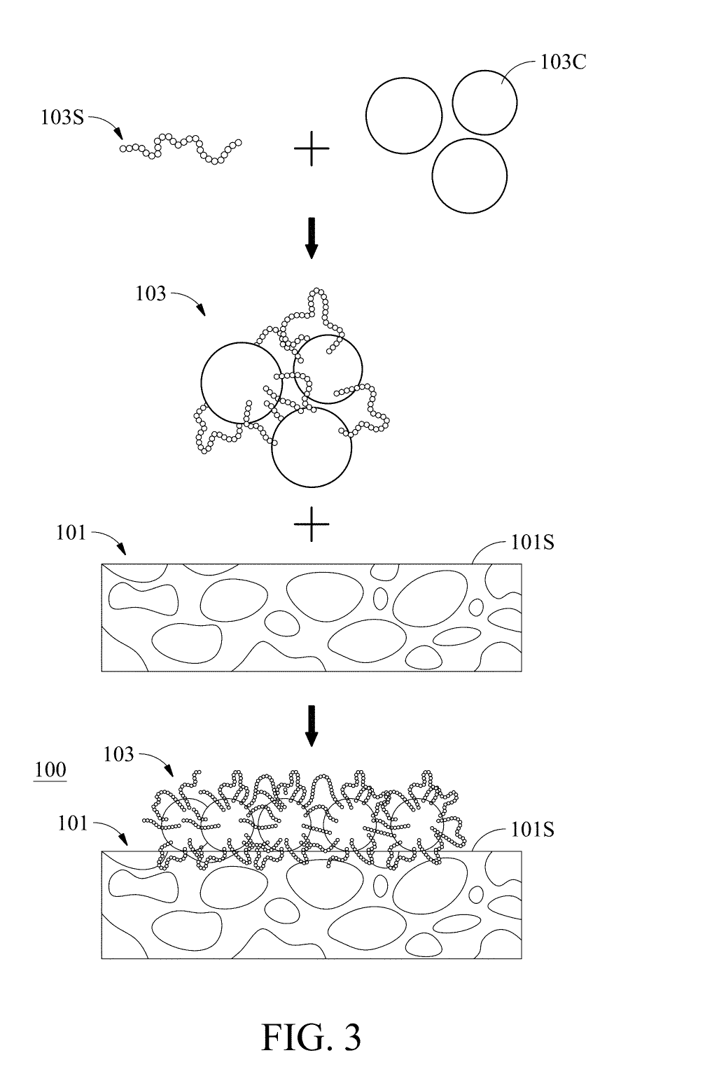

FIG. 3 is a schematic diagram showing one embodiment of a mode for forming the test strip for microorganism detection 100 of the present disclosure shown in FIG. 1B. First, a plurality of colored particles 103C are contacted with a plurality of specific peptides 103S to form a microorganism detection composition 103. Next, the microorganism detection composition 103 is contacted with the porous substrate 101 to make the microorganism detection composition 103 adhere to the surface 101S of the porous substrate 101. The manner of bringing the microorganism detection composition 103 into contact with the porous substrate 101 is not particularly limited, as long as the microorganism detection composition 103 can adhere to the surface of the porous substrate 101. For example, the microorganism detection composition 103 can be caused to make contact with the porous substrate 101 by way of inkjet, spotting, soaking, etc. to make the microorganism detection composition 103 adhere to the surface of the porous substrate 101, but it is not limited thereto.

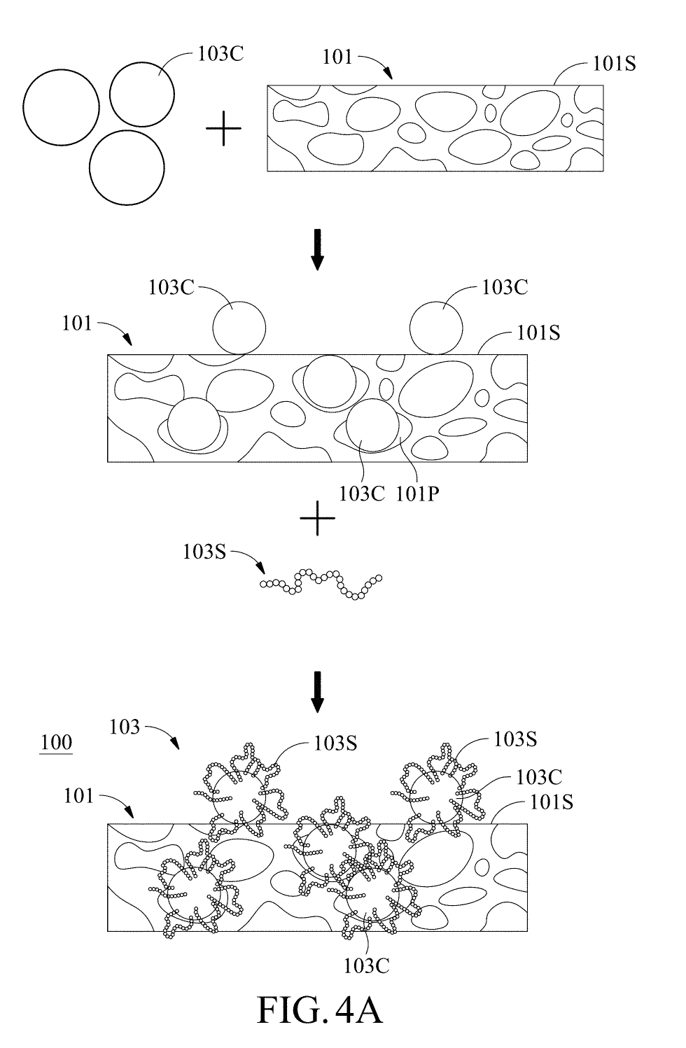

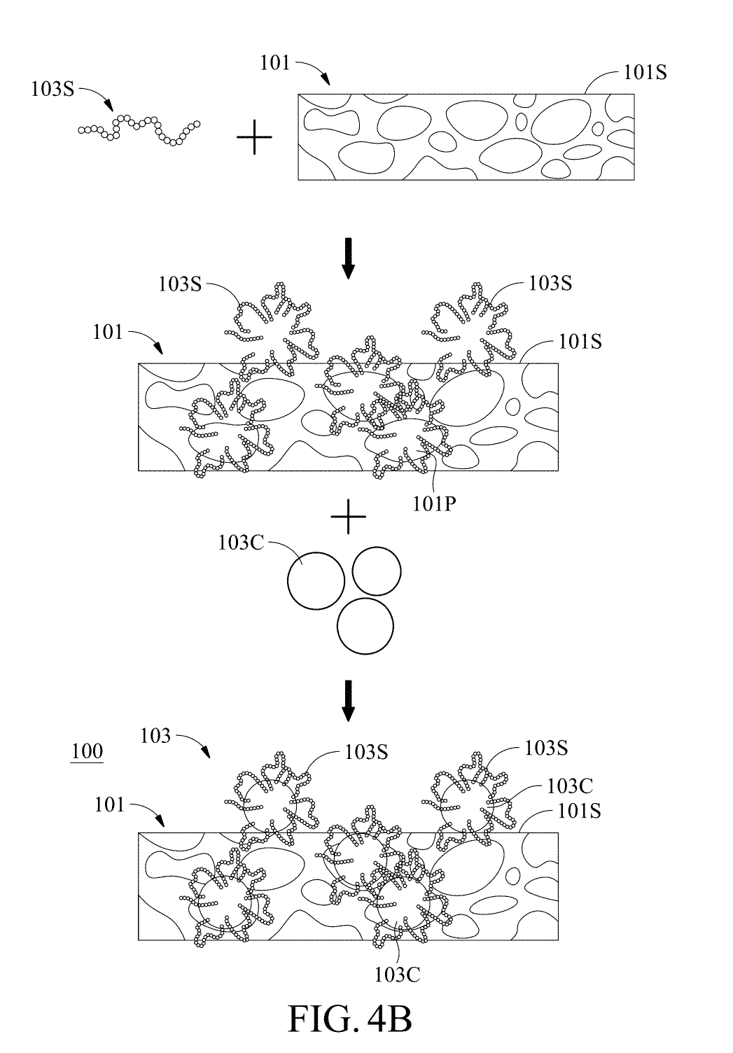

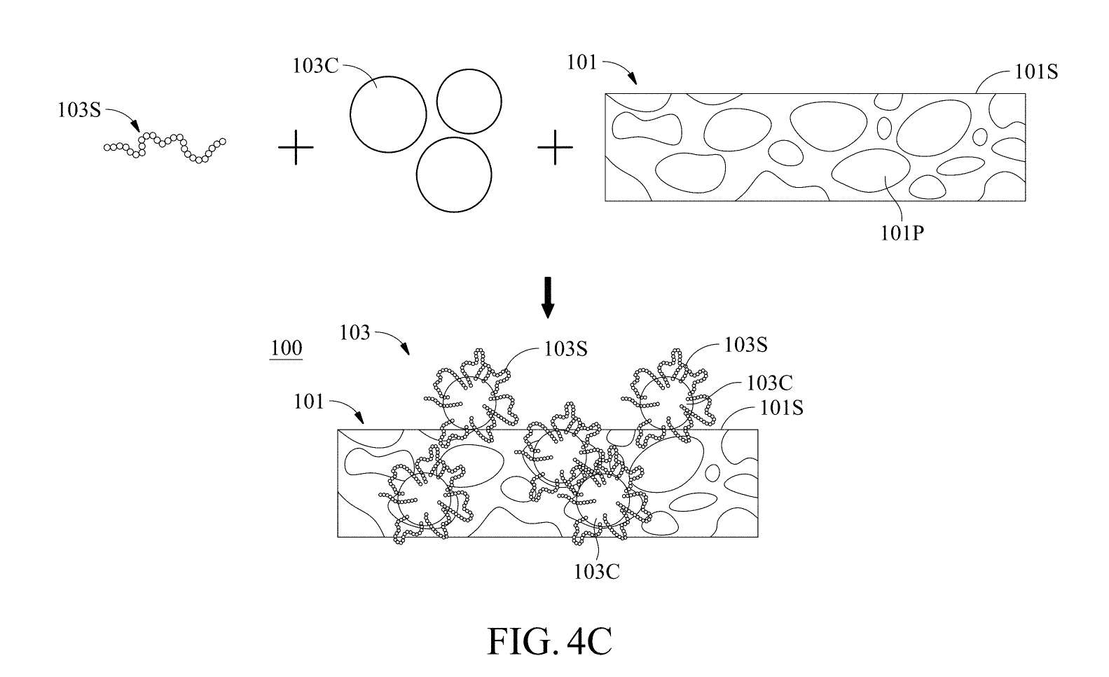

FIGS. 4A-4C are schematic diagrams showing different embodiments of a mode for forming the test strip for microorganism detection 100 of the present disclosure shown in FIG. 1C.

Refer to FIG. 4A and 1C. In the embodiment of FIG. 4A, first, the plurality of colored particles 103C are contacted with the porous substrate 101 to make the plurality of colored particles 103C attach to the surface 101S of the porous substrate 101 and enter the pores 101P of the porous substrate 101. The manner of bringing the plurality of colored particles 103C into contact with the porous substrate 101 is not particularly limited, as long as it enables the plurality of colored particles 103C to attach to the surface 101S of the porous substrate 101 and/or enter the pores 101P of the porous substrate 101. For example, the plurality of colored particles 103C can be caused to make contact with the porous substrate 101 by way of inkjet, spotting or soaking, etc. to make the plurality of colored particles 103C adhere to the surface 101S of the porous substrate 101 and/or enter the pores 101P of the porous substrate 101, but it is not limited thereto. After that, the porous substrate 101 is contacted with the plurality of specific peptides 103S to make the plurality of specific peptides 103S also attach to the surface 101S of the porous substrate 101 and the pores 101P of the porous substrate 101 and to immobilize the plurality of specific peptides 103S on the surfaces of the plurality of colored particles 103C to make the microorganism detection composition 103 adhere to the surface 101S and the interior 101I of the porous substrate 101. Moreover, after the aforementioned step of bringing the plurality of colored particles 103C into contact with the porous substrate 101, the manner of bringing the plurality of specific peptides 103S into contact with the porous substrate 101 is also not particularly limited, as long as it enables the plurality of specific peptides 1035 to attach to the surface 101S of the porous substrate 101 and/or enter the pores 101P of the porous substrate 101, and connect to the plurality of colored particles 103C. For example, the plurality of specific peptides 103S can be caused to make contact with the porous substrate 101 by way of inkjet, spotting or soaking, etc. to make the plurality of specific peptides 103S adhere to the surface 101S of the porous substrate 101 and/or enter the pores 101P of the porous substrate 101, but it is not limited thereto.

Furthermore, refer to FIG. 4B and 1C. In the embodiment of FIG. 4A, first, the plurality of specific peptides 103S are contacted with the porous substrate 101 to make the plurality of specific peptides 103S attach to the surface 101S of the porous substrate 101 and enter the pores 101P of the porous substrate 101. The manner of bringing the plurality of specific peptides 103S into contact with the porous substrate 101 is not particularly limited, as long as it enables the plurality of specific peptides 103S to attach to the surface 101S of the porous substrate 101 and/or enter the pores 101P of the porous substrate 101. For example, the plurality of specific peptides 103S can be caused to make contact with the porous substrate 101 by way of inkjet, spotting or soaking, etc. to make the plurality of specific peptides 103S adhere to the surface 101S of the porous substrate 101 and/or enter the pores 101P of the porous substrate 101, but it is not limited thereto. After that, the porous substrate 101 is contacted with the plurality of colored particles 103C to make the plurality of colored particles 103C also attach to the surface 101S of the porous substrate 101 and enter the pores 101P of the porous substrate 101 and to immobilize the plurality of specific peptides 103S on the surfaces of the plurality of colored particles 103C to make the microorganism detection composition 103 adhere to the surface 101S and the interior 101I of the porous substrate 101. Moreover, after the aforementioned step of causing the plurality of specific peptides 103S to come into contact with the porous substrate 101, the manner of bringing the plurality of colored particles 103C into contact with the porous substrate 101 is also not particularly limited, as long as it enables the plurality of colored particles 103C to attach to the surface 101S of the porous substrate 101 and/or enter the pores 101P of the porous substrate 101, and connect to the plurality of specific peptides 103S. For example, the plurality of colored particles 103C can be caused to make contact with the porous substrate 101 by way of inkjet, spotting or soaking, etc. to make the plurality of colored particles 103C adhere to the surface 101S of the porous substrate 101 and/or enter the pores 101P of the porous substrate 101, but it is not limited thereto.

In addition, refer to FIG. 4C and 1C. In the embodiment of FIG. 4C, first, the plurality of colored particles 103C, the plurality of specific peptides 103S and the porous substrate 101 are contacted with each other at the same time to make the plurality of colored particles 103C and the plurality of specific peptides 103S attach to the surface 101S of the porous substrate 101 and enter the pores 101P of the porous substrate 101 and to immobilize the plurality of specific peptides 103S on the surfaces of the plurality of colored particles 103C to make the microorganism detection composition 103 adhere to the surface 101S and the interior 101I of the porous substrate 101. The manner of making the plurality of colored particles 103C, the plurality of specific peptides 103S and the porous substrate 101 comes into physical contact with each other at the same time is not particularly limited, as long as it enables the plurality of colored particles 103C and the plurality of specific 103 S to attach to the surface 101S of the porous substrate 101 and/or enter the pores 101P of the porous substrate 101. For example, the plurality of colored particles 103C, the plurality of specific peptides 103S and the porous substrate 101 can be soaked in a liquid at the same time to make the plurality of colored particles 103C and the plurality of specific peptides 103S adhere to the surface 101S of the porous substrate 101 and/or enter the pores 101P of the porous substrate 101 and connect to each other, but it is not limited thereto.

The shape of the colored particle 103C in the microorganism detection composition 103 has not particular limitation, for example, the shape of the colored particle may comprise a spherical shape, a square shape, a tapered shape, a rod shape, a polygonal shape, or the like, but is not limited thereto. In one embodiment, the shape of the colored particle may be a spherical shape.

Furthermore, the material of the colored particles 103C in the microorganism detection composition 103 is also not particularly limited, as long as it is not easily dissolved in an aqueous solution. Examples of the material of the colored particle 103C may comprise, but are not limited to a magnetic substance, glass, a polymer material, plastics, nano particles, combinations thereof and the like. In one embodiment, the material of the colored particles may be a magnetic substance so that it can be separated from the test sample and/or recovered by simple magnetic adsorption in the subsequent step.

In addition, the particle size of the colored particle 103C in the microorganism detection composition 103 mentioned may be about 0.01 μm-1 μm, such as about 0.01 μm-0.1 μm, 0.1 μm-0.5 μm, 0.5 μm-1 μm, etc., but it is not limited thereto.

Furthermore, in one embodiment, the colored particles 103C in the microorganism detection composition 103 may be subjected to a chemical treatment so that the specific peptide 103S in the microorganism detection composition 103 can be easily fixed thereon. The foregoing chemical treatment may enable the surface of the colored particle 103C mentioned above be modified with at least one functional group, but is not limited thereto. The type of at least one functional group modified on the surface of the colored particle 103C mentioned above is not particularly limited, and may depend on needs. For example, the type of at least one functional group modified on the surface of the above colored particle 103C may depend on the modification desired to be performed on the specific peptide 103S to be immobilized on the colored particle 103C. Examples of the at least one functional group mentioned above may comprise —COOH, —NH₂, —SH, and the like, but are not limited thereto. In one embodiment, the surface of the foregoing colored particle is modified with —COOH.

In addition, in the aforementioned microorganism detection composition 103, the specific peptide 103S is designed for the specific microorganism to be detected. The foregoing specific microorganism has the ability to cleave this specific peptide. For example, the specific microorganism to be detected can produce a specific enzyme which can be a secreted or membrane protein form, and the specific peptide 103S mentioned above is designed for this specific enzyme. Specifically, by designing a specific cleavage site for the specific enzyme in the above-mentioned specific peptide 103S, the above-specific peptide mentioned can be specifically recognize and cut by the specific enzyme produced by the specific microorganism.

With regard to the usage scenario for the test strip for the microorganism detection 100 of present disclosure, please refer to FIG. 5. Before a detection for a testing sample is performed (referring to the left side of FIG. 5), since the plurality of colored particles 103C in the microorganism detection composition 103 are connected to each other by the plurality of peptides 1035 and aggregate, the microorganism detection composition 103 covering at least one part 101S-1 of the surface 101S of the porous substrate 101 makes part 101S-1 of the surface 101S of the test strip for microorganism detection 100 present the second color mentioned above (i.e. the color of the plurality of colored particles 103C) (referring to the small box of the left side of FIG. 5), and when the specific microorganism exists in the test sample, the specific microorganism will cleave the plurality of specific peptides 103S in the microorganism detection composition 103 which make the plurality of colored particles 103C be connected to each other, so that the aggregation of the plurality of colored particles 103C is reduced or even the plurality of colored particles 103C do not aggregate any more, and this cause that the above second color (i.e. the color of the plurality of colored particles 103C) presented on the part 101S-1 of the surface 101S of the porous substrate 101 becomes light and/or the part 101S-1 of the surface 101S of the porous substrate 101 is exposed to present the first color of the porous substrate 101 (referring to the small box of the right side of FIG. 5).

In the aforementioned microorganism detection composition 103, the length of the specific peptide 103S may be about 10-200 amino acids, such as about 10-150 amino acids, 10-100 amino acids, about 20-50 amino acids, about 15-25 amino acids, about 25-35 amino acids, about 30-40 amino acids, but it is not limited thereto.

In one embodiment, the N-terminal end and the C-terminal end of the specific peptide 103S may be respectively modified with a first functional group and a second functional group to make the specific peptide be easily immobilized on the surface of the colored particle 103C. The first functional group and the second functional group mentioned above may be homo-functional functional groups and the two may be the same or different while the first functional group and the second functional group mentioned above also may be hetero-functional functional groups. The types of the first functional group and the second functional group are not particularly limited and may depend on needs. For example, the type of the first functional group and the second functional group depend on the modification desired to be performed on the colored particle 103C. For instance, the first functional group and the second functional group may independently comprise —NH₂, —COOH, biotin, —SH, etc., but they are not limited thereto. In a specific embodiment, the first functional group and the second functional group may both be —NH₂.

In one embodiment, the test strip for microorganism detection 100 of the present disclosure is used for detecting the presence of a lactic acid bacterium. The lactic acid bacterium may comprise, but is not limited to Lactobacillus reuteri, Lactobacillus pentosus, Lactobacillus gasseri, Lactobacillus salivarius, Lactobacillus paracasei, Lactobacillus plantarum, any combination thereof, etc.

In the foregoing embodiment in which the test strip for microorganism detection 100 of the present disclosure is used for detecting the presence of a lactic acid bacterium, the sequence of the specific peptide 1035 of the microorganism detection composition 103 may comprise at least a part of the sequence of casein.

Moreover, in the foregoing embodiment in which the test strip for microorganism detection 100 of the present disclosure is used for detecting the presence of a lactic acid bacterium, the sequence of the specific peptide 1035 of the microorganism detection composition 103 may comprise a sequence having at least about 75% sequence identity to the amino acid sequence of SEQ ID NO. 1, SEQ ID NO. 2, SEQ ID NO. 3, SEQ ID NO. 4 or SEQ ID NO. 5. For example, the sequence of the specific peptide 103S of the microorganism detection composition 103 may comprise the amino acid sequence of SEQ ID NO. 1, SEQ ID NO. 2, SEQ ID NO. 3, SEQ ID NO. 4 or SEQ ID NO. 5, but it is not limited thereto.

“At least about 75% sequence similarity” as described herein means that there is at least about 75% sequence similarity between two sequences, such as 80%, 81%, 82%, 85%, 90%, 95%, 99%, 99.5%, 100% sequence similarity, but it is not limited thereto.

In the foregoing embodiment in which the test strip for microorganism detection 100 of the present disclosure is used for detecting the presence of a lactic acid bacterium, in one specific embodiment, the sequence of the specific peptide 1035 of the microorganism detection composition 103 may comprise the amino acid sequence of SEQ ID NO. 1. Moreover, in this specific embodiment, the sequence of the specific peptide 103S mentioned above may be the amino acid sequence of SEQ ID NO. 1 or SEQ ID NO. 6.

Furthermore, in the foregoing embodiment in which the test strip for microorganism detection 100 of the present disclosure is used for detecting the presence of a lactic acid bacterium, in one specific embodiment, the sequence of the specific peptide 103S of the microorganism detection composition 103 may comprise the amino acid sequence of SEQ ID NO. 2. Moreover, in this specific embodiment, the sequence of the specific peptide 103S mentioned above may be the amino acid sequence of SEQ ID NO. 2 or SEQ ID NO. 7.

Furthermore, in the foregoing embodiment in which the test strip for microorganism detection 100 of the present disclosure is used for detecting the presence of a lactic acid bacterium, in one specific embodiment, the N-terminal end and the C-terminal end of the specific peptide 103S of the microorganism detection composition 103 are both modified with —NH₂ group. Moreover, the surface of the colored particle 103C of the microorganism detection composition 103 in the test strip for microorganism detection 100 is modified with —COOH group, and the colored particle 103C is a magnetic bead. In addition, the material of the porous substrate 101 of the test strip for microorganism detection 100 is nitrocellulose.

The present disclosure also provides a microorganism detection method. The microorganism detection method of the present disclosure is used for detecting the presence of a specific microorganism.

Examples of the specific microorganism mentioned above may comprise a bacterium, a fungi, etc., but they are not limited thereto. The bacterium may comprise, but is not limited to a lactic acid bacterium, Escherichia coli, a bacterium belonging to the genus Salmonella, Staphylococcus aureus, a bacterium belonging to the genus Listeria or a bacterium belonging to the genus Shigella, etc. Moreover, examples of the lactic acid bacterium mentioned above may comprise Lactobacillus reuteri, Lactobacillus pentosus, Lactobacillus gasseri, Lactobacillus salivarius, Lactobacillus paracasei, Lactobacillus plantarum, any combination thereof, but they are not limited thereto.

The microorganism detection method of the present disclosure may comprise the following steps, but it is not limited thereto.

A test sample is contacted with any test strip for microorganism detection 100 of the preset disclosure.

The test sample is not particularly limited, as long as it is an object that may contain microorganisms. In one embodiment, the test sample may comprise foods, such as fresh food, canned food, packaged beverage, etc., but is not limited thereto.

The terms “contact”, “contacting”, “is contacted to” or “in contact with” in the present disclosure do not mean that the test sample must be in direct contact with the test strip for microorganism detection 100 of the present disclosure, as long as a scenario in which the test strip for microorganism detection 100 of the present disclosure can react with the test sample and/or an enzyme released by a bacterium in the test sample can be achieved. In one embodiment, the test sample may be a meat stored at a low temperature, and the test strip for microorganism detection 100 of the present disclosure and the meat stored at a low temperature are packaged together in a sealed package, and that enables the test strip for microorganism detection 100 to react with the test sample and/or an enzyme released by a bacterium in the test sample without direct contact of the meat with the test for microorganism detection 100 of the present disclosure.

There is no particular limitation in the time needed for the test sample to be in contact with the test strip for microorganism detection 100 of the present disclosure, as long as the test sample can be sufficiently reacted with the test strip for microorganism detection 100 of the present disclosure. At room temperature (about 20-28° C.), the time during which the test sample should be in contact with the test strip for microorganism detection 100 of the present disclosure may be about 7 days or less, such as about 6 hours to 7 days, about 6-12 hours, 3-7 days, about 8 hours, about 12 hours, about 1 day, about 5 days, about 6 days, about 7 days, etc., but it is not limited thereto. In a low temperature refrigerated condition (about 2-8° C.), the time during which the test sample should be in contact with the test strip for microorganism detection 100 of the present disclosure may be about 3 weeks or less, such as about 12 hours to 3 weeks, about 12 to 48 hours, about 1-3 weeks, about 18 hours, about 24 hours, about 1 week, about 2 weeks, about 3 weeks, etc., but it is not limited thereto.

After the test sample comes into contact with the test strip for microorganism detection, the color change of the test strip for microorganism detection is determined.

FIG. 5 is a schematic diagram showing the detection of a specific microorganism in a test sample by the microorganism detection method of the present disclosure.

As mentioned above, since the specific peptide 103S of the microorganism detection composition 103 in the test strip for microorganism detection 100 is designed for a specific microorganism desired to be detected, during detection of a specific microorganism by the microorganism detection method of the present disclosure, if the specific microorganism to be detected exists in the test sample, the specific microorganism will cleave the plurality of specific peptides 103S which connect the plurality of colored 103C to each other in the microorganism detection composition 103 of the test strip for microorganism detection 100, so that the aggregation of the plurality of colored particles 103C in the microorganism detection composition 103 is reduced or even the plurality of colored particles 103C do not aggregate any more in the microorganism detection composition 103, and this cause that the above second color (i.e. the color of the plurality of colored particles 103C) presented on the part 101S-1 of the surface 101S of the porous substrate 101 becomes light and/or the part 101S-1 of the surface 101S of the porous substrate 101 is exposed to present the first color of the porous substrate 101 (referring to the small box of the right side of FIG. 5). On the contrary, if the microorganism to be detected does not exist in the test sample, the plurality of colored particles 103C in the microorganism detection composition 103 of the test strip for microorganism detection 100 of the present disclosure will still maintain aggregation so that the second color (i.e. the color of the plurality of colored particles 103C) is still maintained on the part 101S-1 of the surface 101S of the porous substrate 101 of the test strip for microorganism detection 100 of the present disclosure.

Since the microorganism detection method of the present disclosure only needs to use the test strip for microorganism detection 100 of the present disclosure, and can quickly determine the presence or absence of a specific microorganism by only visual inspection, it has the advantages of convenience and ease of operation.

Moreover, in the microorganism detection method of the present disclosure, the foregoing test sample may be in a liquid form, but is not limited thereto.

Furthermore, there is no particular limitation in the temperature at which the test sample should be in contact with the test strip for microorganism detection 100 of the present disclosure, as long as the specific microorganism being detected can react with the specific peptide 103S in the test strip for microorganism detection 100 of the present In one embodiment, the test sample comes into contact with the test strip for microorganism detection 100 at a temperature of about 2-56° C., such as about 4-10° C., about 10-20° C., about 20-40° C., about 40-56° C., etc., but it is not limited thereto. In one specific embodiment, the contact of the test sample with the test strip for microorganism detection 100 is performed at about 4° C., 25° C., 37° C. or 56° C.

In one embodiment, the microorganism detection method is used for detecting the presence of a lactic acid bacterium. The lactic acid bacterium may comprise, but is not limited to Lactobacillus reuteri, Lactobacillus pentosus, Lactobacillus gasseri, Lactobacillus salivarius, Lactobacillus paracasei, Lactobacillus plantarum, any combination thereof, etc.

In the foregoing embodiment in which the microorganism detection method is used for detecting the presence of a lactic acid bacterium, it is needed to use a test strip for microorganism detection 100 of the present disclosure suited for the detection of lactic acid bacteria.

In the foregoing embodiment in which the microorganism detection method is used for detecting the presence of a lactic acid bacterium, with regard to the test strip for microorganism detection 100 suited for the detection of lactic acid bacteria, the sequence of the specific peptide 103S of the microorganism detection composition 103 may comprise at least a part of the sequence of casein.

Moreover, in the foregoing embodiment in which the microorganism detection method is used for detecting the presence of a lactic acid bacterium, with regard to the test strip for microorganism detection 100 suited for the detection of lactic acid bacteria, the sequence of the specific peptide 103S of the microorganism detection composition 103 may comprise a sequence having at least about 75% sequence identity to the amino acid sequence of SEQ ID NO. 1, SEQ ID NO. 2, SEQ ID NO. 3, SEQ ID NO. 4 or SEQ ID NO. 5. For example, the sequence of the specific peptide 1035 of the microorganism detection composition 103 may comprise the amino acid sequence of SEQ ID NO. 1, SEQ ID NO. 2, SEQ ID NO. 3, SEQ ID NO. 4 or SEQ ID NO. 5, but it is not limited thereto.

“At least about 75% sequence similarity” as described herein means that there is at least about 75% sequence similarity between two sequences, such as 75%, 80%, 81%, 82%, 85%, 90%, 95%, 99%, 99.5%, 100% sequence similarity, but it is not limited thereto.

In the foregoing embodiment in which the microorganism detection method is used for detecting the presence of a lactic acid bacterium, in one specific embodiment, with regard to the test strip for microorganism detection 100 suited for the detection of lactic acid bacteria, the sequence of the specific peptide 103S of the microorganism detection composition 103 may comprise the amino acid sequence of SEQ ID NO. 1. Moreover, in this specific embodiment, the sequence of the specific peptide 103S mentioned above may be the amino acid sequence of SEQ ID NO. 1 or SEQ ID NO. 6.

Furthermore, in the foregoing embodiment in which the microorganism detection method is used for detecting the presence of a lactic acid bacterium, with regard to the test strip for microorganism detection 100 suited for the detection of lactic acid bacteria, in one specific embodiment, the sequence of the specific peptide 103S of the microorganism detection composition 103 may comprise the amino acid sequence of SEQ ID NO. 2. Moreover, in this specific embodiment, the sequence of the specific peptide 103S mentioned above may be the amino acid sequence of SEQ ID NO. 2 or SEQ ID NO. 7.

### EXAMPLES

1. Example 1: Peptide Designing

Lactic acid bacteria were set as the detection targets, the following 7 specific peptides of lactic acid bacteria were designed according to the sequence of casein, and Mission Biotech (Taiwan) and Bio Pioneer Tech CO., LTD (Taiwan) were entrusted to assist in the synthesis of these 7 specific peptides of lactic acid bacteria.

(1) For α-casein

α1-23-mer peptide (SEQ ID NO. 1); α1-35-mer peptide (SEQ ID NO. 6).

(2) For β-casein

β2-23-mer peptide (SEQ ID NO. 2); β2-35-mer peptide (SEQ ID NO. 7); β3-23-mer peptide (SEQ ID NO. 3); β4-23-mer peptide (SEQ ID NO. 4); β5-23-mer peptide(SEQ ID NO. 5).

2. Example 2: Selection for Substrate

Photocopy paper, filter paper (Whatman), nitrocellulose film (GE) and PVDF film (Millipore) were tested as porous substrates, and a magnetic bead suspension with a magnetic bead content of 7.5 mg/ml was prepared.

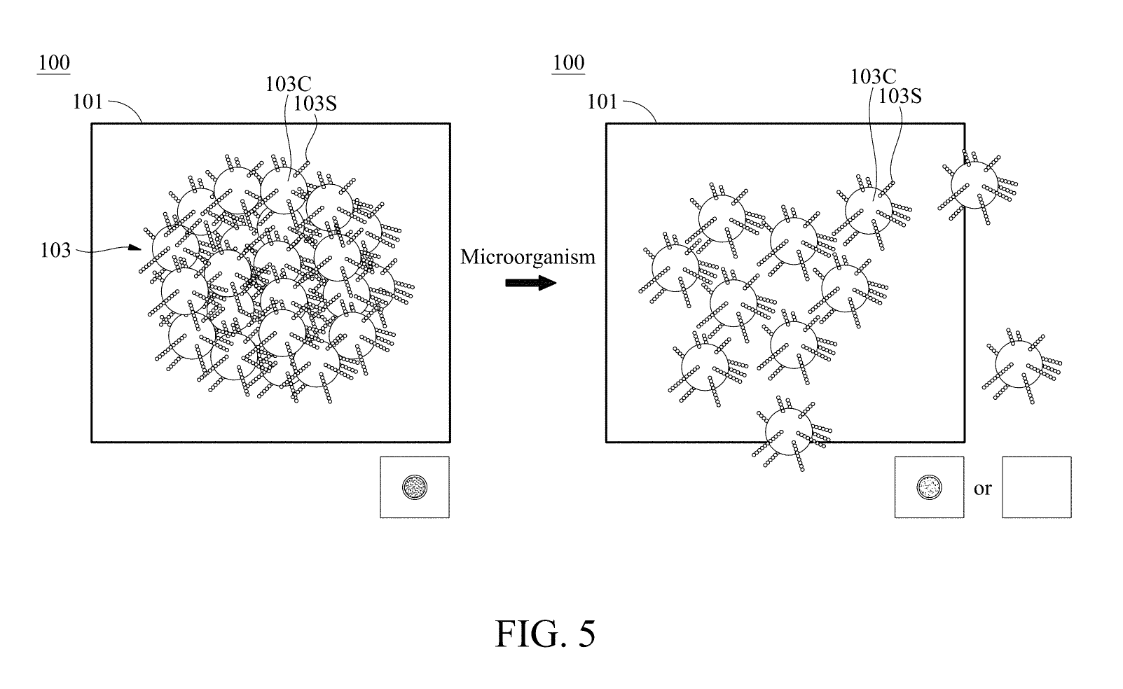

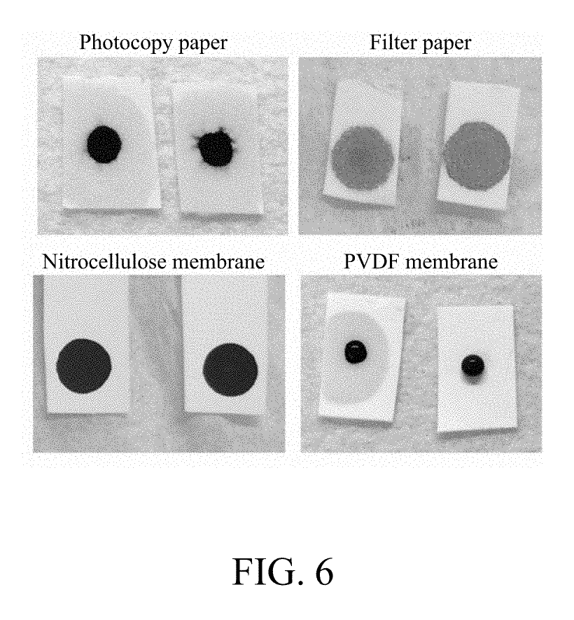

The magnetic bead suspension with a volume of 10 μlwas dropped on the photocopy paper, the filter paper, the nitrocellulose film or the PVDF film, and the results are as shown in FIG. 6.

Based on FIG. 6, it can be known that after the magnetic bead suspension is dropped on the aforementioned substrates, the magnetic bead suspension can be diffused into a complete circular shape on the nitrocellulose film and the magnetic beads can be uniformly distributed on the nitrocellulose film. Moreover, although the magnetic bead suspension did not diffuse into a complete circle on the photocopy paper, the magnetic beads still could be uniformly distributed on the photocopy paper.

Overall, for all of the potential substrate materials that were tested, nitrocellulose film had the best diffusion performance. Since nitrocellulose film has properties such as porosity, negative charge, easy adsorption of protein and peptides, and is often used as a substrate material in many medical test strips, the nitrocellulose film was as the ideal material for use in the subsequent experiments.

Example 3: Preparation of Test Strip for Microorganism Detection

3.1 Method A

3.1.1 Preparation of test strip for microorganism detection containing magnetic beads on which peptides without modification are immobilized

Magnetic beads activated by 1-ethyl-3-(3-dimethyl aminopropyl) carbodiimide hydrochloride/N-Hydroxysuccinimide (EDC/NHS) were mixed with the peptides (α1-23-mer peptide or β2-23-mer peptide), and then the mixture was dropped on a nitrocellulose film

3.1.2 Preparation of Test Strip for Microorganism Detection Containing Magnetic Beads on Which Peptides with Modification are Immobilized

Magnetic beads activated by EDC/NHS were mixed with the peptides modified with NH₂ functional group (α1-23-mer peptide, β2-23-mer peptide, α1-35-mer peptide or β2-35-mer peptide of which the two ends were modified with NH₂ functional group) (α1-23-mer peptide, β2-23-mer peptide, α1-35-mer peptide and β2-35-mer peptide of which the two ends were modified with NH₂ functional group are also called α1-23-mer-NH₂ peptide, β2-23-mer-NH₂ peptide, α1-35-mer-NH₂ peptide and β2-35-mer-NH₂ peptide in the following, respectively), and then the mixture was dropped on a nitrocellulose film.

3.2 Method B

Preparation of test strip for microorganism detection containing magnetic beads on which peptides with modification are immobilized.

Magnetic beads activated by EDC/NHS, peptides modified with NH₂ functional group (α1-23-mer-NH₂ peptide or β2-23-mer-NH₂ peptide) and a nitrocellulose film were brought into contact with each other at the same time and left to stand overnight. After that, a film full of magnetic beads was obtained.

4. Example 4

Scanning electron microscopy (SEM) analysis for test strip for microorganism detection

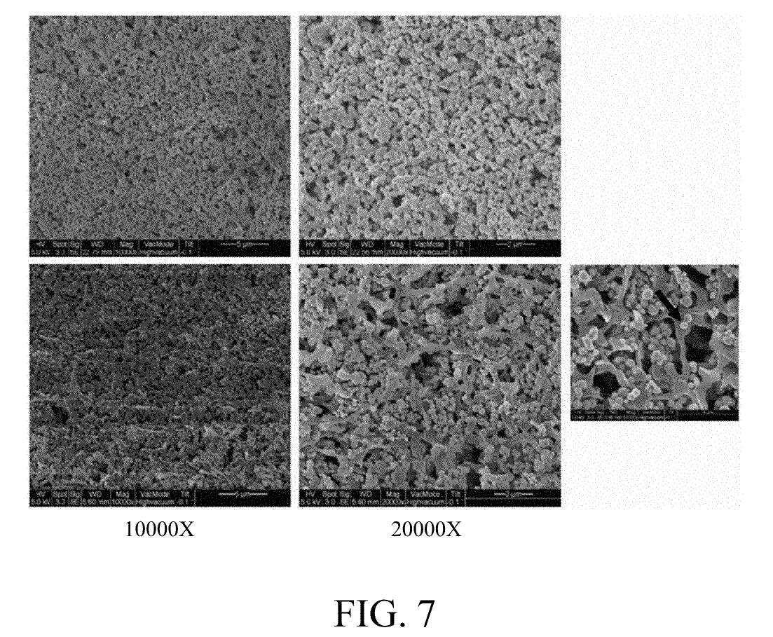

The test strips respectively obtained by Method A and Method B in Example 3 were analyzed by a scanning electron microscope, respectively, and the results are as shown in FIG. 7.

The upper block of FIG. 7 shows the result of scanning electron microscopy analysis of the test strip obtained by Method A (containing α1-23-mer-NH₂ peptide) while the lower block of FIG. 7 shows the result of scanning electron microscopy analysis of the test strip obtained by Method B (containing α1-23-mer-NH₂ peptide), wherein the magnification of the photograph on the left side is 10000× while the magnification of the photograph on the right side is 20000×.

According to the photograph on the upper block of FIG. 7, it can be known that the surface of the test strip obtained by Method A is covered with a layer of magnetic beads. The photographs on the lower block of FIG. 7 clearly show that magnetic beads can enter the pores of the nitrocellulose film in the test strip obtained by Method B.

5. Example 5: Enzymatic Degradation Test for Test Strip for Microorganism Detection Containing Magnetic Beads on Which Peptides Without Modification are Immobilized

Enzymatic degradation tests were performed on the test strips obtained by item 3.1.1 in Method A of Example 3 (containing magnetic beads on which α1-23-mer peptide or β2-23-mer peptide without modification was immobilized) by proteinase K or trypsin with different dilution ratios. PBS buffer was used in the control group.

Image analysis was performed on the test strips before and after the test, and the image intensity (gray density) for the determining region was calculated (the value of all black was 255; the lighter the color was, the lower the value was; the value for all white was 0). Digestion level (DL) of peptide was calculated based on the image intensity. Calculation formula for digestion level of peptide is as shown in the following:

Digestion level (DL)=(Image intensity of the region covered by the magnetic beads before the test−Image intensity of the region covered by the magnetic beads after the test)²/Area of the region covered by the magnetic beads before the test.

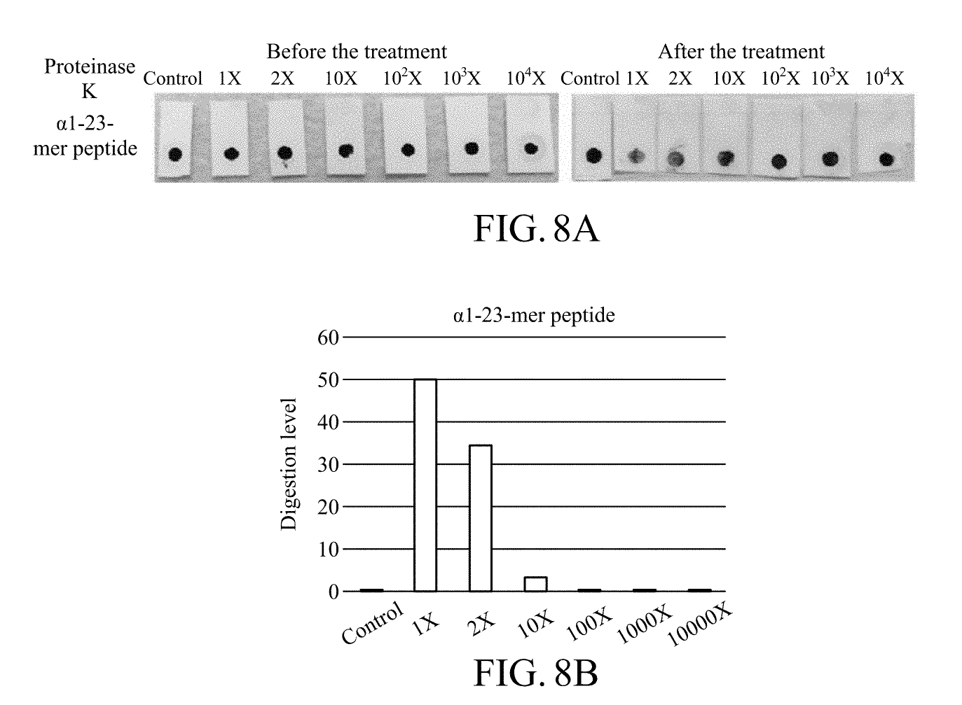

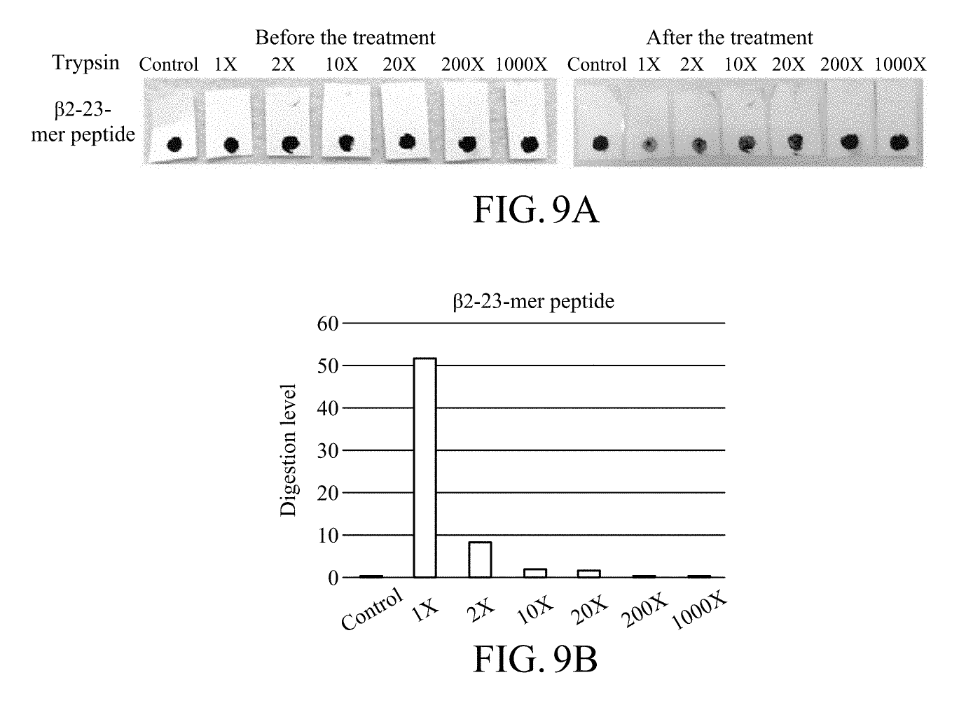

The results are as shown in FIGS. 8A and 8B and FIGS. 9A and 9B. FIGS. 8A and 8B show the results of the test strip containing magnetic beads on which α1-23-mer peptide without modification is immobilized while FIGS. 9A and 9B show the results of the test strips containing magnetic beads on which β2-23-mer peptide without modification is immobilized.

Based on FIG. 8A, it can be known that as the dilution ratios of proteinase K increases, the stripping degree of the magnetic bead from the test strip containing magnetic beads on which α1-23-mer peptide without modification is immobilized relatively decreases, and the result of analyzing the image intensity with ImageJ software is the same as the visual result. Moreover, based on FIG. 8B, it can be known that as the dilution ratios of proteinase K increases, the digestion level of peptide relatively decreases. The results mentioned above represent that the stripping of the magnetic beads on which α1-23-mer peptide without modification is immobilized is caused by proteinase K.

Similarly, FIG. 9A shows that as the dilution ratios of trypsin increases, the stripping degree of the magnetic bead from the test strip containing magnetic beads on which β2-23-mer peptide without modification is immobilized relatively decreases, and FIG. 9B shows that as the dilution ratios of trypsin increases, the digestion level of peptide relatively decreases. The results mentioned above also represent that the stripping of the magnetic beads on which β2-23-mer peptide without modification is immobilized is caused by trypsin.

6. Example 6: Lactic Acid Bacteria Test for Test Strip for Microorganism Detection Containing Magnetic Beads on Which Peptides Without Modification are Immobilized

The test strips obtained by item 3.1.1 in Method A of Example 3 (containing magnetic beads on which α1-23-mer peptide or β2-23-mer peptide without modification was immobilized) were tested by six kinds of lactic acid bacteria (Lactobacillus reuteri, Lactobacillus pentosus, Lactobacillus gasseri, Lactobacillus salivarius, Lactobacillus paracasei or Lactobacillus plantarum). Each test was performed in duplication. PBS buffer was used in the control group.

The test strips were co-cultured with lactic acid bacteria in a 37° C. incubator.

After 3 days, the test strip was taken out and photographed, image analysis was performed by ImageJ software, and the image intensity (gray density) for the determining region was calculated (the value of all black was 255; the lighter the color was, the lower the value was; the value for all white was 0). Digestion level (DL) of peptide was calculated based on the image intensity. Three parameters, digestion level (DL) of peptide, area change (AC) of the region covered by the magnetic beads, and detachment level were calculated. Calculation formula for digestion level of peptide is as shown above while calculation formula for area change (AC) of the region covered by the magnetic beads and formula for detachment level are as shown in the following.

Area change (AC) of the region covered by the magnetic beads=[(Area of the region covered by the magnetic beads before the test−Area of the region covered by the magnetic beads after the test)/Area of the region covered by the magnetic beads before the test]×100;

Detachment level=Digestion level (DL) of peptide×Area change (AC) of the region covered by the magnetic beads.

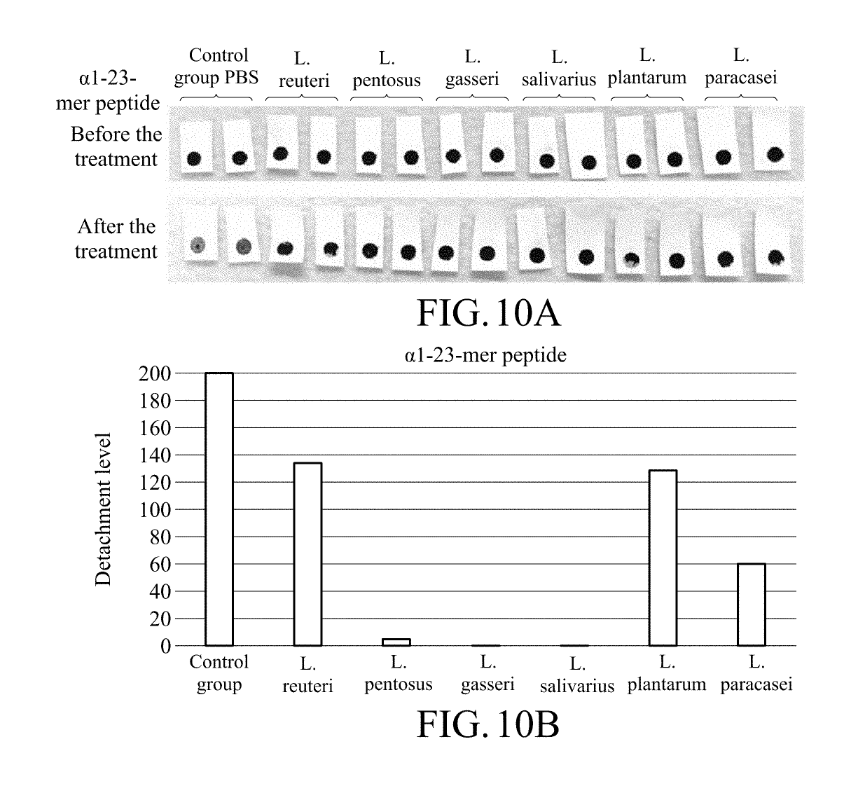

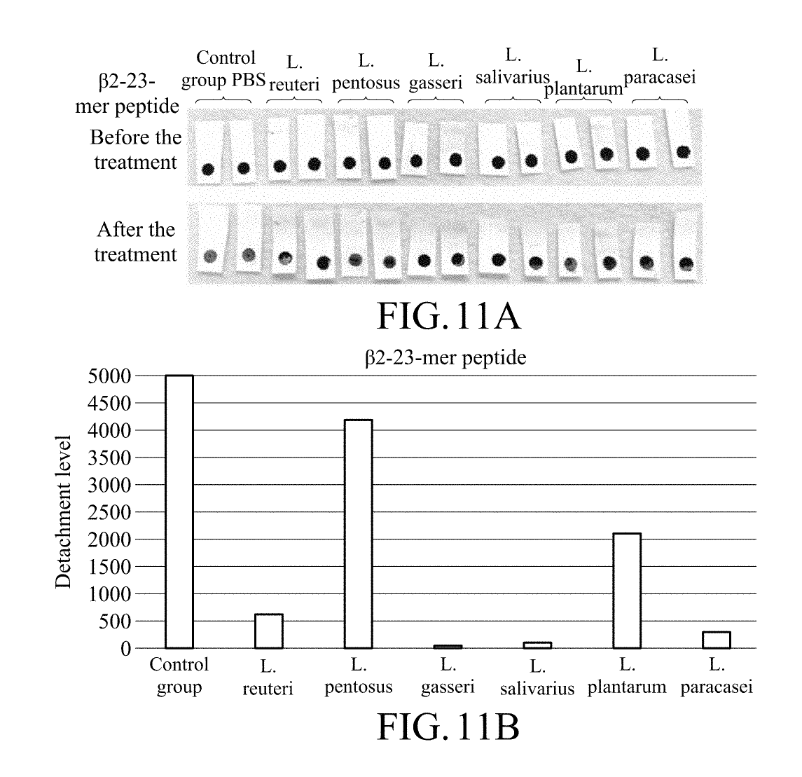

The results are as shown in FIGS. 10A and 10B and FIGS. 11A and 11B. FIGS. 10A and 10B show the test results for the test strip for microorganism detection containing magnetic beads on which α1-23-mer peptide without modification was immobilized, and FIGS. 11A and 11B show the test results for the test strip for microorganism detection containing magnetic beads on which β2-23-mer peptide without modification was immobilized.

Based on FIGS. 10A and 10B, it can be known that Lactobacillus reuteri, Lactobacillus plantarum and Lactobacillus paracasei prefer α1-23-mer peptide. Based on FIGS. 11A and 11B, it can be known that Lactobacillus plantarum and Lactobacillus pentosus prefer β2-23-mer peptide. Lactobacillus plantarum, Lactobacillus paracasei, Lactobacillus pentosus and Lactobacillus reuteri were selected as the material for the subsequent tests.

Example 7: Enzymatic Degradation Test for Test Strip for Microorganism Detection Containing Magnetic Beads on Which Peptides with Modification are Immobilized

Enzymatic degradation tests were performed on the test strips obtained by item 3.1.2 in Method A and Method B of Example 3 (containing magnetic beads on which α1-23-mer-NH₂ peptide, β2-23-mer-NH₂ peptide, α1-35-mer-NH₂ peptide or β2-35-mer-NH₂ peptide was immobilized) by proteinase K or trypsin with different dilution ratios. Control group: PBS buffer.

Image analysis was performed on the test strips before and after the test, and two parameters, digestion level of peptide and area change (AC) of the region covered by the magnetic beads, were calculated. Calculation formulas for digestion level of peptide and area change (AC) of the region covered by the magnetic beads are shown as above.

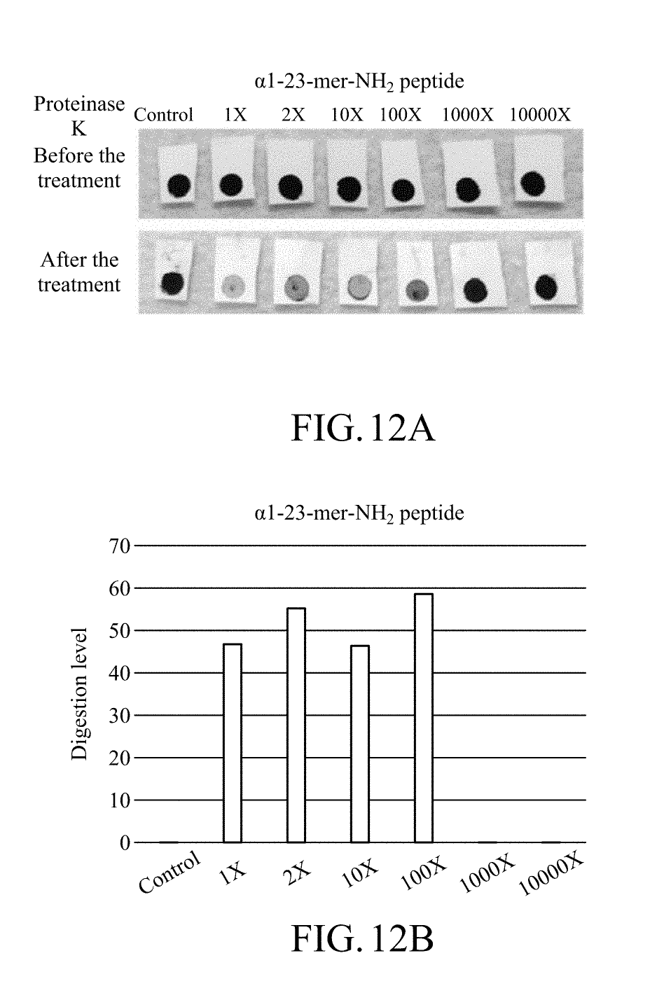

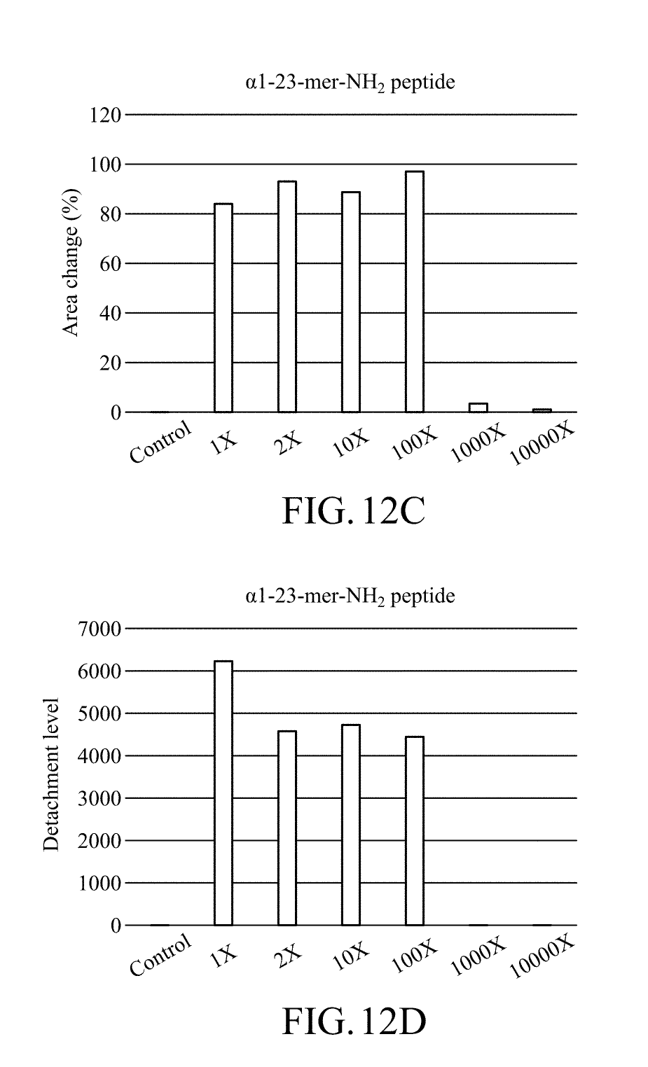

The results are as shown in FIGS. 12A to 12D, FIGS. 13A to 13D, FIG. 14, FIG. 15, FIGS. 16A and 16B and FIGS. 17A and 17B.

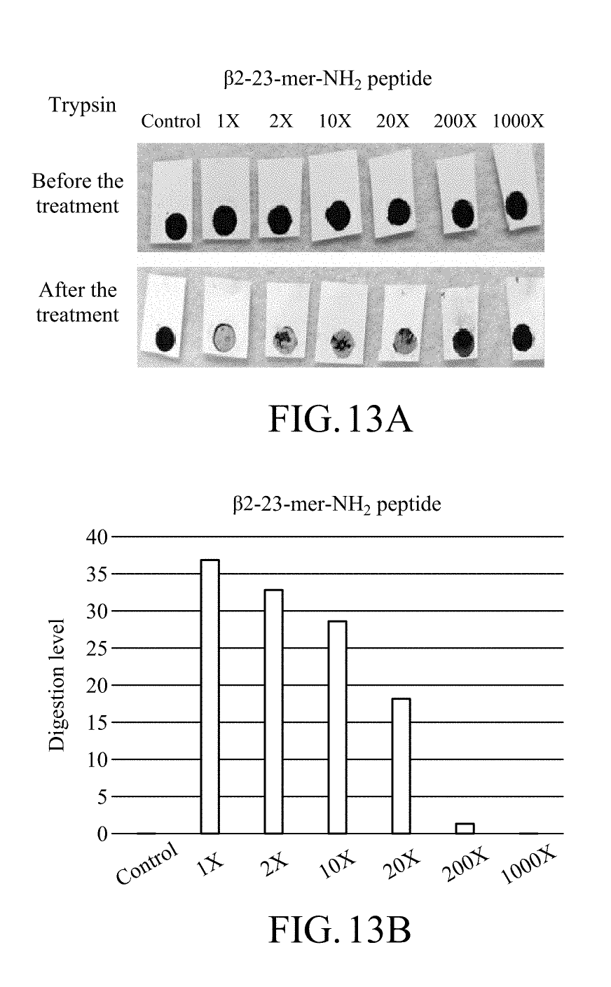

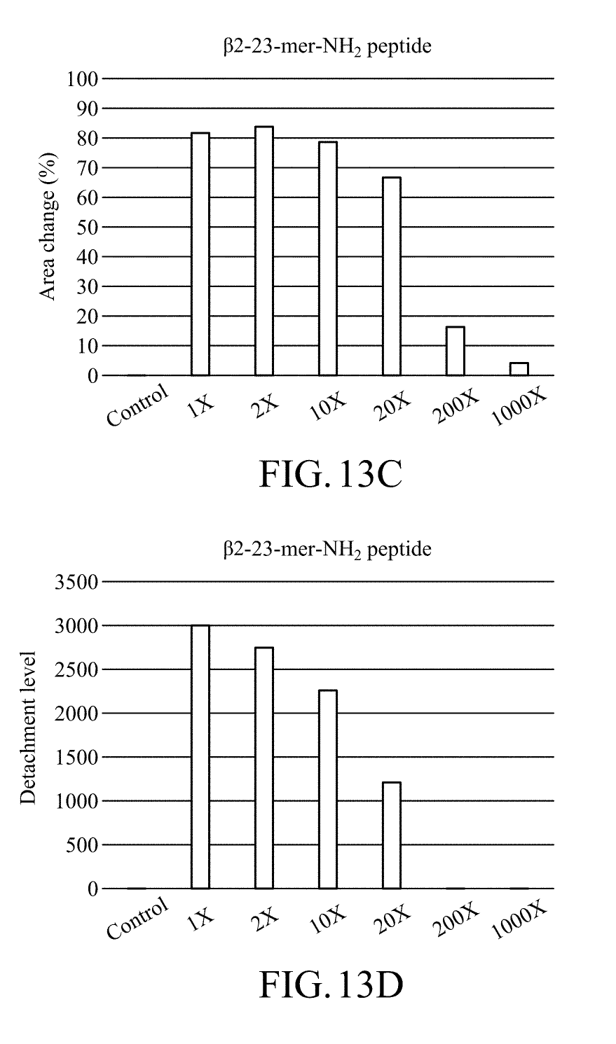

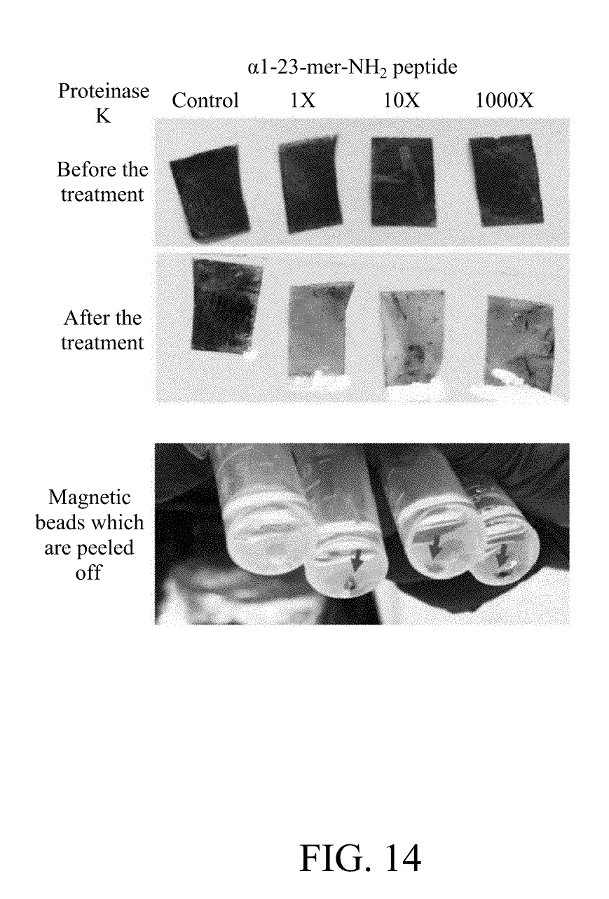

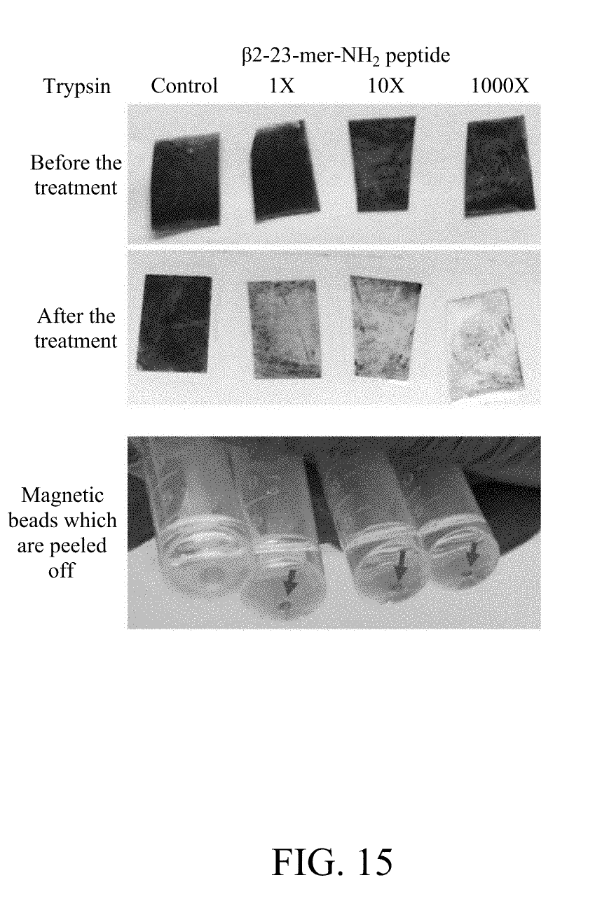

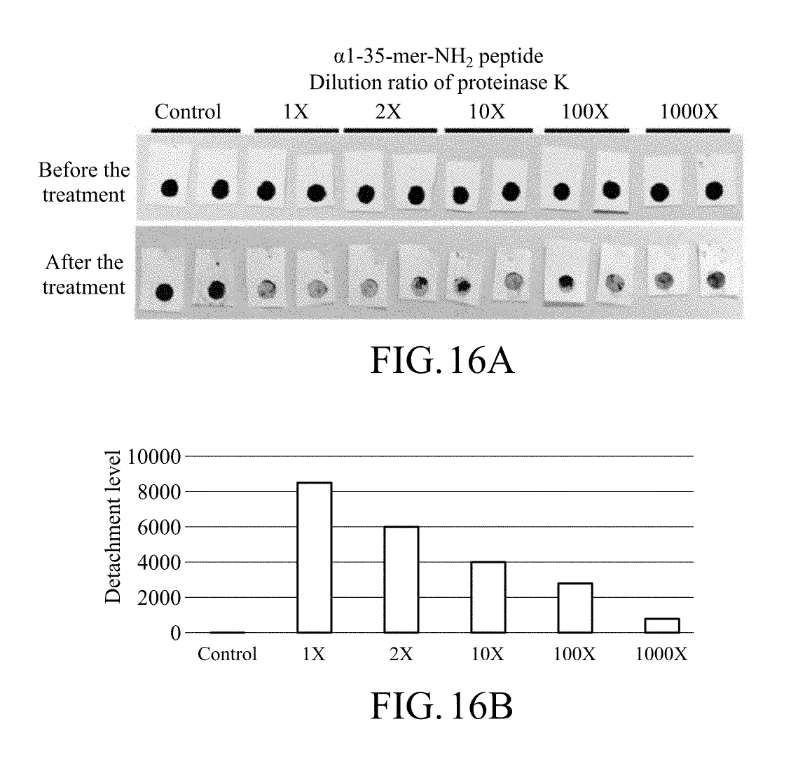

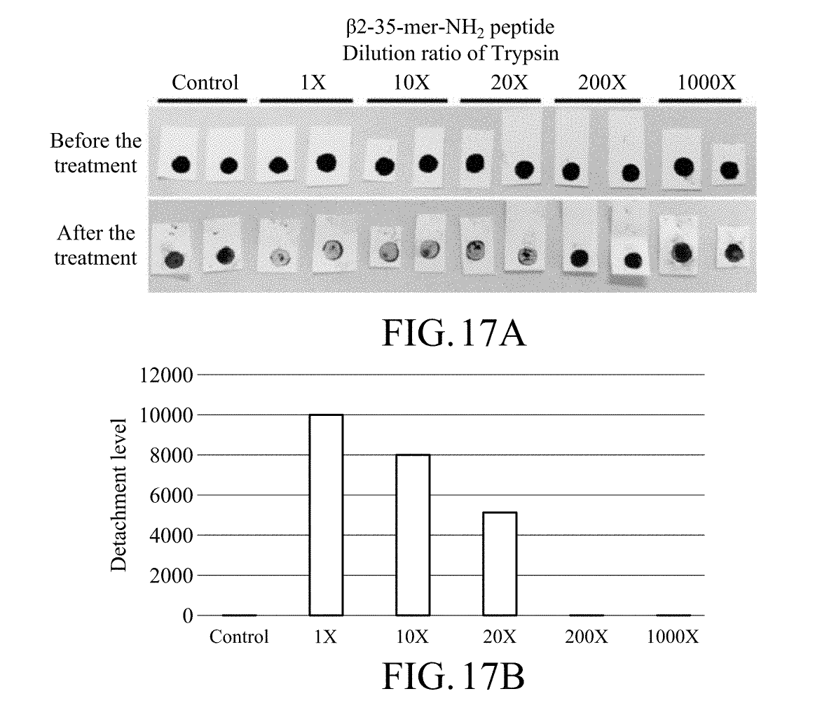

FIGS. 12A to 12D show the test results for the test strip containing magnetic beads on which α1-23-mer-NH₂ peptide prepared by Method A was immobilized while FIGS. 13A to 13D show the test results for the test strip containing magnetic beads on which β2-23-mer-NH₂ peptide prepared by Method A was immobilized. FIG. 14 shows the test results for the test strip containing magnetic beads on which α1-23-mer-NH₂ peptide prepared by Method B was immobilized while FIG. 15 shows the test results for the test strip containing magnetic beads on which β2-23-mer-NH₂ peptide prepared by Method B was immobilized. FIGS. 16A and 16B show the test results for the test strip containing magnetic beads on which α1-35-mer-NH₂ peptide prepared by Method A was immobilized while FIGS. 17A and 17B show the test results for the test strip containing magnetic beads on which β2-35-mer-NH₂ peptide prepared by Method A was immobilized.

Based on FIGS. 12A to 12D, it can be known that as the dilution ratio of proteinase K increases, the stripping degree of the magnetic bead from the test strip containing magnetic beads on which α1-23-mer-NH₂ peptide prepared by Method A was immobilized relatively decreases and the digestion level of peptide relatively decreases, and that represents that the stripping of the magnetic beads is caused by proteinase K. According to FIGS. 16A and 16B, it can be known that the test strip containing magnetic beads on which α1-35-mer-NH₂ peptide prepared by Method A was immobilized has a results similar to that of the test strip containing magnetic beads on which α1-23-mer-NH₂ peptide prepared by Method A was immobilized mentioned above.

In addition, based on FIGS. 13A to 13D, it can be known that as the dilution ratio of trypsin increases, the stripping degree of the magnetic bead from the test strip containing magnetic beads on which β2-23-mer-NH₂ peptide prepared by Method A was immobilized relatively decreases and the digestion level of peptide relatively decreases, and that represents that the stripping of the magnetic beads is caused by trypsin. According to FIGS. 17A and 17B, it can be known that the test strip containing magnetic beads on which β2-35-mer-NH₂ peptide prepared by Method A was immobilized has a results similar to that of the test strip containing magnetic beads on which β2-23-mer-NH₂ peptide prepared by Method A was immobilized mentioned above.

Furthermore, based on FIG. 14, it can be known that as the dilution ratio of proteinase K increases, the stripping degree of the magnetic bead from the test strip containing magnetic beads on which α1-23-mer-NH₂ peptide prepared by Method B was immobilized relatively decreases, and that represents that the stripping of the magnetic beads is caused by proteinase K. Similarly, based on FIG. 15, it can be known that as the dilution ratio of trypsin increases, the stripping degree of the magnetic bead from the test strip containing magnetic beads on which β2-23-mer-NH₂ peptide prepared by Method B was immobilized relatively decreases, and that represents that the stripping of the magnetic beads is caused by trypsin.

Example 8: Lactic Acid Bacteria Test at Low Temperature for Test Strip for Microorganism Detection Containing Magnetic Beads on Which Peptides with Modification are Immobilized

At 4° C. (temperature for simulating the storage ambient of vacuum-packed meat), the test strips obtained by item 3.1.2 of Example 3 (containing magnetic beads on which α1-23-mer-NH₂ peptide or β2-23-mer-NH₂ peptide was immobilized) were tested by different lactic acid bacteria. M.R.S. medium was used in the control group.

The test strip was co-cultured with lactic acid bacteria in a 4° C. refrigerator for 2 days. The strip was taken out and photographed every day and image analysis was performed by ImageJ. Three parameters, digestion level (DL) of peptide, area change (AC) of the region covered by the magnetic beads and detachment level, were calculated. Calculation formula for digestion level of peptide, area change (AC) of the region covered by the magnetic beads, and detachment level are shown as above.

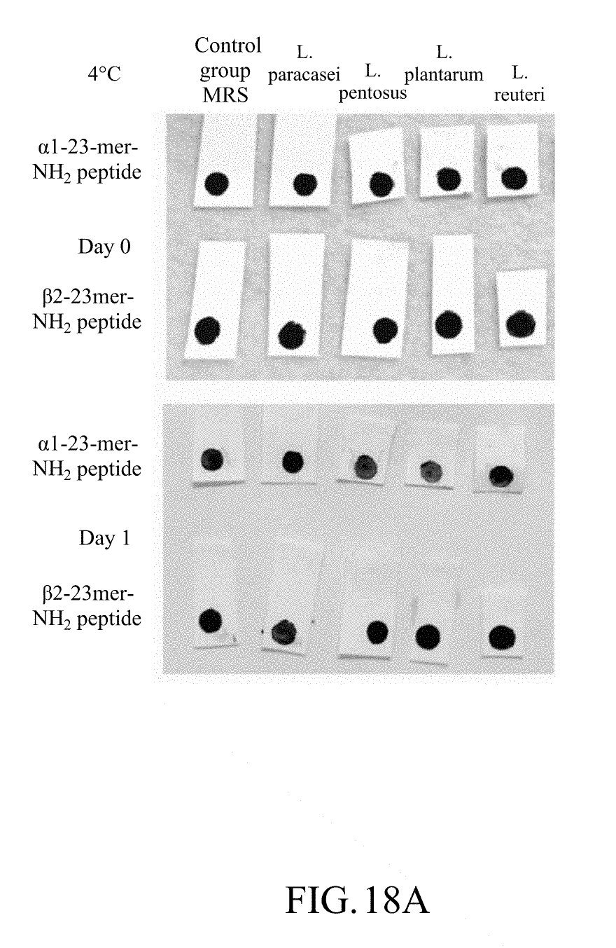

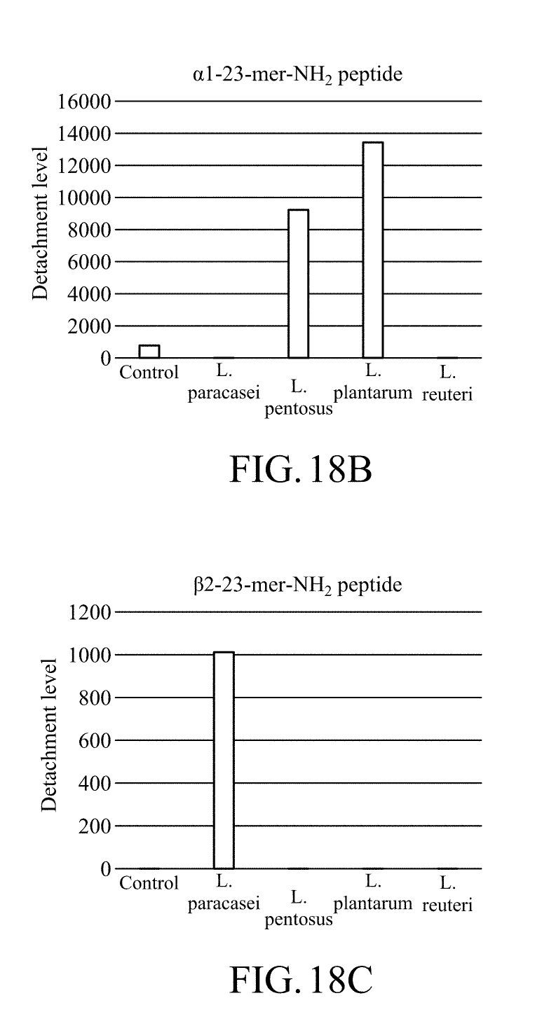

The results are as shown in FIGS. 18A to 18C.

FIGS. 18A to 18C show test results for the test strips containing magnetic beads on which α1-23-mer-NH₂ peptide or β2-23-mer-NH₂ peptide was immobilized.

According to FIGS. 18A to 18C, it can be known that the test strip of the present disclosure can detect lactic acid bacteria at a low temperature, and different lactic acid bacteria have their preference, in which the α1-23-mer-NH₂ peptide can be decomposed by more kinds of lactic acid bacteria. For the test strip containing magnetic beads on which α1-23-mer-NH₂ peptide was immobilized, on Day 1 in the low-temperature culturing, it could react with Lactobacillus plantarum and Lactobacillus pentosus, and the stripping of the magnetic beads could be observed, and on Day 2, except that no obvious stripping of the magnetic beads was observed in the co-culture group with Lactobacillus paracasei, the stripping of the magnetic beads was observed in the co-culture groups with other three strains (Lactobacillus plantarum, Lactobacillus pentosus and Lactobacillus reuteri), and that represented that the test strip was capable of detecting the bacterial species with which it was co-cultured. As for the test strip containing magnetic beads on which β2-23-mer-NH₂ peptide was immobilized, it could react with Lactobacillus paracasei on Day 2.

Example 9

Confirmation of the minimum detectable amount of lactic acid bacteria for test strip

The test strips containing magnetic beads on which α1-23-mer-NH₂ peptide obtained by item 3.1.2 of Example 3 was immobilized was test by Lactobacillus plantarum. Positive control: proteinase K; negative control: MRS medium.

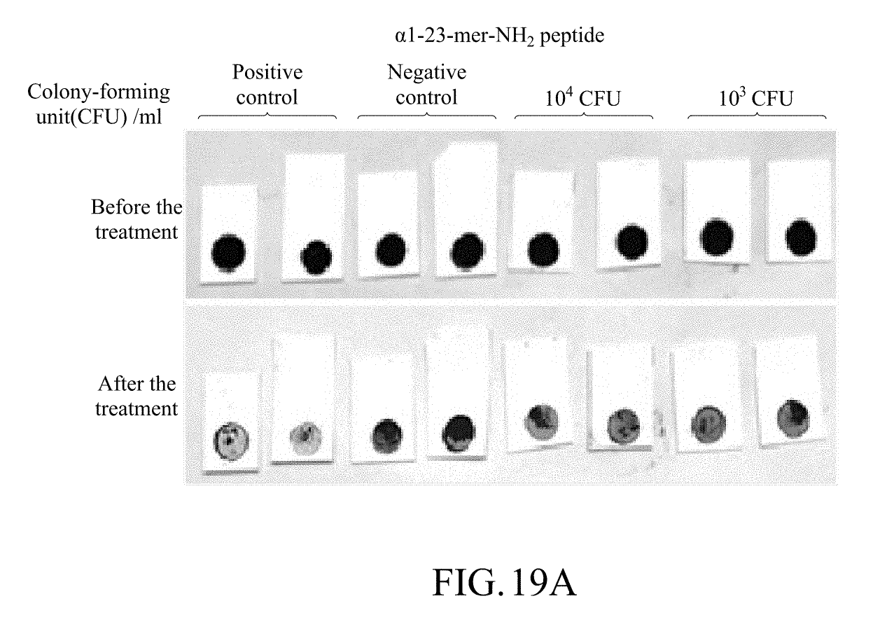

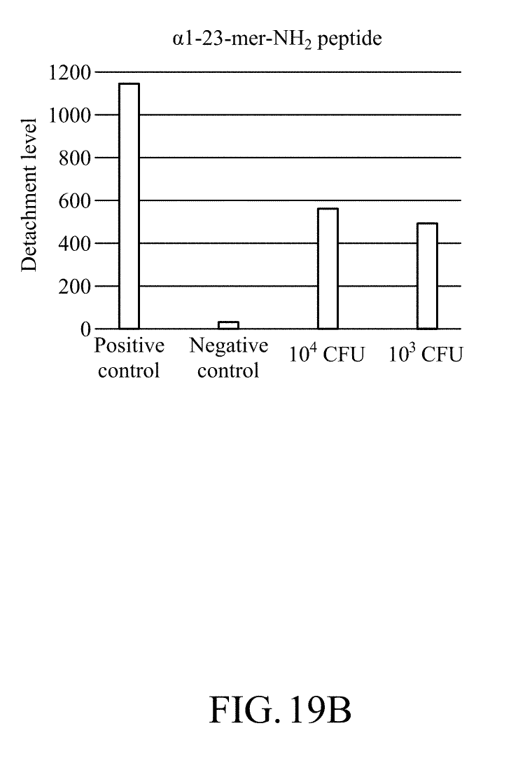

10³ or 10⁴ colony-forming unit (cfu) of bacteria was co-cultured with the test at 4° C. for 18 hours. The results are as shown in FIGS. 19A and 19B.

FIG. 19A shows a photograph for co-culturing the test strips containing magnetic beads on which α1-23-mer-NH₂ peptide was immobilized with the Lactobacillus plantarum with different amount of bacteria in a confirmation test for the minimum detectable amount of Lactobacillus plantarum for the test strip. FIG. 19B shows the detachment level of the test strips containing magnetic beads on which α1-23-mer-NH₂ peptide was immobilized in a confirmation test for the minimum detectable amount of Lactobacillus plantarum for the test strip.

According to FIGS. 19A and 19B, it can be known that the test strip can detect bacteria in the group with the initial bacterial amount of 10³ and 10⁴ colony forming units.

According to the above results, it can be known that the test strip of the present disclosure can effectively detect the target bacteria at a low bacterial amount (10³ colony forming units), and thus the test strip of the present disclosure has high sensitivity and can be effectively applied to detection of microorganisms in food, and can be used for microorganism detection at low temperatures.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A test strip for microorganism detection, comprising:
a porous substrate, a surface of which has a first color; and
a microorganism detection composition which is at least adhered to a part of a surface of the porous substrate or at least adhered to a part of a surface of the porous substrate and a part of an interior of the porous substrate, comprising:
a plurality of colored particles having a second color that is different than the first color; and
a plurality of specific peptides immobilized on the surfaces of the plurality of colored particles, in which the plurality of specific peptides connect the plurality of colored particles to each other to make the plurality of colored particles aggregate to cover the part of the surface of the porous substrate and present the second color on the part of the surface of the porous substrate,
wherein the specific peptide is designed for a specific microorganism, and the specific microorganism has an ability to cleave the specific peptide.

2. The test strip for microorganism detection as claimed in claim 1, wherein a material for the porous substrate comprises a natural fiber material or an artificial fiber material.

3. The test strip for microorganism detection as claimed in claim 1, wherein the surface of the colored particle is modified with at least one functional group.

4. The test strip for microorganism detection as claimed in claim 3, wherein the at least one functional group comprises —COOH, —NH₂ or —SH.

5. The test strip for microorganism detection as claimed in claim 1, wherein the length of the specific peptide is 10-200 amino acids.

6. The test strip for microorganism detection as claimed in claim 1, wherein the N-terminal end and the C-terminal end of the specific peptide are respectively modified with a first functional group and a second functional group, and wherein the first functional group and the second functional group are homogeneous functional groups.

7. The test strip for microorganism detection as claimed in claim 1, wherein the microorganism comprises a bacterium or a fungi.

8. The test strip for microorganism detection as claimed in claim 7, wherein the bacterium comprises a lactic acid bacterium, Escherichia coli, a bacterium belonging to the genus Salmonella, Staphylococcus aureus, a bacterium belonging to the genus Listeria or a bacterium belonging to the genus Shigella.

9. The test strip for microorganism detection as claimed in claim 8, wherein the lactic acid bacterium comprises Lactobacillus reuteri, Lactobacillus pentosus, Lactobacillus gasseri, Lactobacillus salivarius, Lactobacillus paracasei and/or Lactobacillus plantarum.

10. The test strip for microorganism detection as claimed in claim 1, wherein the specific microorganism is a lactic acid bacterium.

11. The test strip for microorganism detection as claimed in claim 10, wherein the sequence of the specific peptide comprises a sequence having at least about 80% sequence identity to the amino acid sequence of SEQ ID NO. 1, SEQ ID NO. 2, SEQ ID NO. 3, SEQ ID NO. 4 or SEQ ID NO. 5.

12. The test strip for microorganism detection as claimed in claim 11, wherein the sequence of the specific peptide is the amino acid sequence of SEQ ID NO. 1 or SEQ ID NO. 6.

13. The test strip for microorganism detection as claimed in claim 11, wherein the sequence of the specific peptide is the amino acid sequence of SEQ ID NO. 2 or SEQ ID NO. 7.

14. A microorganism detection method, comprising:
bringing a test sample into contact with the test strip for microorganism detection as claimed in claim 1; and
determining the color change of the test strip for microorganism detection after the test sample comes into contact with the test strip for microorganism detection,
wherein when the test sample contains the specific microorganism, the specific microorganism cleaves the specific peptide, and that leads to the plurality of colored particles no longer aggregating to lighten the second color, which is present due to the aggregation of the plurality of colored particles, and/or to expose part of the surface of the porous substrate, thereby making the first color visible.

15. The microorganism detection method as claimed in claim 14, wherein the microorganism comprises a bacterium or a fungi.

16. The microorganism detection method as claimed in claim 15, wherein the bacterium comprises a lactic acid bacterium, Escherichia coli, a bacterium belonging to the genus Salmonella, Staphylococcus aureus, a bacterium belonging to the genus Listeria or a bacterium belonging to the genus Shigella.

17. The microorganism detection method as claimed in claim 16, wherein the lactic acid bacterium comprises Lactobacillus reuteri, Lactobacillus Lactobacillus gasseri, Lactobacillus salivarius, Lactobacillus paracasei and/or Lactobacillus plantarum.

18. The microorganism detection method as claimed in claim 14, wherein the sequence of the specific peptide comprises a sequence having at least about 80% sequence identity to the amino acid sequence of SEQ ID NO. 1, SEQ ID NO. 2, SEQ ID NO. 3, SEQ ID NO. 4 or SEQ ID NO. 5.

19. The microorganism detection method as claimed in claim 18, wherein the sequence of the specific peptide is the amino acid sequence of SEQ ID NO. 1 or SEQ ID NO. 6.

20. The microorganism detection method as claimed in claim 18, wherein the sequence of the specific peptide is the amino acid sequence of SEQ ID NO. 2 or SEQ ID NO. 7.
